(19) United States
(12) Patent Application Publication
(10) Pub. No.: US 2006/0116765 A1
(43) Pub. Date: Jun. 1, 2006

(54) Refractive corrective lens (RCL)

(76) Inventors: Larry W. Blake, Coto De Caza, CA (US); William C. Huddleston, Anaheim Hills, CA (US); Gene Currie, Anaheim Hills, CA (US); Jorge L. Alio, Alicante (ES)

Correspondence Address: KLIMA LAW OFFICES, P.L.L.C., P.O. Box 2855, Stafford, VA, 22555-2855, US

(21) Appl. No.: 11/249,358

(22) Filed: Oct. 14, 2005

Related U.S. Application Data
(63) Continuation-in-part of application No. 09/631,576, filed on Aug. 4, 2000.

(51) Int. Cl. A61F 2/16; B29D 11/00

(52) U.S. Cl. 623/6.46; 623/6.36; 623/6.27; 264/2.5

(57) ABSTRACT

An anterior chamber refractive correction lens (RCL), preferably a custom anterior chamber refractive correction lens (c-RCL) for visual or optical correcting multiple defects or problems of the eye.

### RELATED APPLICATIONS

This is a continuation-in-part (CIP) of U.S. patent application Ser. No. 09/631,576, entitled “TWO PART “L”-SHAPED PHAKIC IOL”, filed on Aug. 4, 2000, fully incorporated herein by reference.

### FIELD OF THE INVENTION

The present invention is directed to an artificial ocular lens (AOL), in particular a refractive correction lens (RCL). A preferred embodiment includes a foldable or deformable anterior chamber phakic refractive correction lens (pRCL) or aphakic refractive correction lens (apRCL) having multiple visual correction features or capabilities or otherwise a custom refractive correction lens (cRCL) configured for implantation through a very small incision in the eye.

### BACKGROUND OF THE INVENTION

The concept of implanting an artificial ocular lens (AOL) into the eye is old. The initial artificial lens was an intraocular lens (IOL) implanted in the eye to replace a damaged natural lens or cataract lens. The early intraocular lens was made of optical glass, and then the glass material was eventually replaced with a rigid optical grade plastic material such as polymethylmethacylate (PMMA). The rigid glass or plastic lens requires a substantially large incision to be made in the patient's eye for implantation therein.

With time, foldable or deformable intraocular lenses (IOLs) were proposed and made for insertion through a small incision in the eye of around three and one-half millimeters (3.5 mm) or less. It was found that the smaller the eye incision was made, the less surgical complications occurred and better the post surgical eye vision was obtained by the patient. Today, incisions on the order of two and one-half millimeters (2.5 mm) to two millimeters (2.0 mm), or less can be used for implanting foldable or deformable intraocular lenses using a forceps or lens injecting device.

Still today, most artificial ocular lenses (AOLs) being surgically implanted in the eye are intraocular lenses (IOLs) for the replacement of the natural lens due to cataract. In some instances, a clear non-cataract natural lens is removed (i.e. clear lensectomy), and replaced with an intraocular lens to improve or correct the patient's vision. The latest developments include phakic refractive correction lenses (pRCLs) such as the Kelman Duet anterior chamber lens for refractive correction of the eye.

The artificial ocular lenses (AOLs) including intraocular lenses (IOLs) and refractive correction lenses (RCLs) currently being made and surgically implanted in the eye today typically have only a single or one (1) visual correction factor or capability to provide power correction of the eye. A limited number of intraocular lenses (IOLs) made and implanted today have two (2) visual correction factors or capabilities for both power correction and astigmatic correction (e.g. toric adjustment) of the eye. The intraocular lens (IOLs) are manufactured in a limited number or increments of lens powers and a limited number or increments of sphere and cylinder for astigmatic correction. The “best fit” and “best available” intraocular lens (IOL) for a particular patient is selected by the eye surgeon after examination of the patient's eye. Specifically, the eye surgeon's choice of intraocular lens is limited to the “best fit” IOL available in the incremental series of both power and toric to best visually correct the patient's eye. Many times the particular intraocular lens selected and implanted by the eye surgeon does not visually correct the patient's eye to a suitable extent or degree.

The refractive correction lenses (RCLs) are still in the early stages of being proposed and in some instances are being researched and developed. Actual manufacturing and implantation of refractive correction lenses (RCLs) is very limited providing little clinical and statistical data. For example, the Kelman Duet anterior chamber phakic refractive correction lens (pRCL) is currently made in very small quantities and implanted in Europe. The Kelman Duet pRCL only provides for refractive correction of the power of the eye. Aphakic refractive correction lenses (apRCLs) have been proposed for use in combination with an artificial capsular lenses or intraocular lenses (IOLs), and may be the same or similar in design or configuration to phakic refractive correction lens (pRCLs).

There exists a need to be able to accurately and effectively visually or optically correct multiple visual defects or problems with a patient's eye using a custom artificial ocular lens (c-AOL) such as a custom intraocular lens (c-IOL) and/or a custom refractive correction lens (c-RCL) such as a custom phakic refractive correction lens (c-pPRCL) or a custom aphakic refractive correction lens (c-apRCL). The artificial ocular lenses (AOLs) according to the present invention address this need.

### SUMMARY OF THE INVENTION

A first (1^{st}) object of the present invention is to provide an improved artificial ocular lens (AOL) for implantation in the eye.

A second (2^{nd}) object of the present invention is to provide an improved intraocular lens (IOL).

A third (3^{rd}) object of the present invention is to provide an improved refractive correction lens (RCL).

A fourth (4^{th}) object of the present invention is to provide an improved phakic refractive correction lens (pRCL).

A fifth (5^{th}) object of the present invention is to provide an improved aphakic refractive correction lens (apRCL).

A sixth (6^{th}) object of the present invention is to provide a custom artificial ocular lens (c-AOL).

A seventh (7^{th}) object of the present invention is to provide a custom intraocular lens (c-IOL).

An eighth (8^{th}) object of the present invention is to provide a custom refractive correction lens (c-RCL).

A ninth (9^{th}) object of the present invention is to provide a custom phakic refractive lens (c-PRL).

A tenth (10^{th}) object of the present invention is to provide a custom aphakic refractive correction lens (c-apRCL).

An eleventh (11^{th}) object of the present invention is to provide an intraocular lens (IOL) configured to visually correct at least three (3) different types of visual defects or problems with a patient's eye.

A twelfth (12^{th}) object of the present invention is to provide a refractive correction lens (RCL) configured to visually correct at least two (2) different types of visual defects or problems with a patient's eye.

A thirteenth (13^{th}) object of the present invention is to provide an anterior chamber refractive correction lens (RCL) configured to visually correct at least two (2) different types of visual defects or problems with a patient's eye.

A fourteenth (14^{th}) object of the present invention is to provide a refractive correction lens (RCL) configured to visually correct at least three (3) different types of visual defects or problems with a patient's eye.

A fifteenth (15^{th}) object of the present invention is to provide an anterior chamber refractive correction lens (RCL) configured to correct at least three (3) different types of visual defects or problems with a patient's eye.

A sixteenth (16^{th}) object of the present invention is to provide an artificial ocular lens (AOL) capable of visually correcting a patient's vision to 20:20 or best correctable vision.

A seventeenth (17^{th}) object of the present invention is to provide an artificial ocular lens (AOL) capable of correcting a patient's vision to 20:10 or best correctable vision.

An eighteenth (18^{th}) object of the present invention is to provide an artificial ocular lens (AOL) capable of correcting a patient's vision to 20:7 or best correctable vision.

A nineteenth (19^{th}) object of the present invention is to provide a refractive correction lens (RCL) configured to correct the power and astigmatism of the eye.

A twentieth (20^{th}) object of the present invention is to provide an anterior chamber refractive correction lens (acRCL) configured to correct the power and astigmatism of the eye.

A twenty-first (21^{st}) object of the present invention is to provide an artificial ocular lens (AOL) configured to correct for aberrations and/or cornea, iris, natural lens, retina and/or other inner eye structure imperfections of the eye.

A twenty-second (22^{nd}) object of the present invention is to provide an artificial ocular lens (AOL) configured to correct for aberrations and/or surface imperfections of the natural lens of the eye.

A twenty-third (23^{rd}) object of the present invention is to provide an artificial ocular lens (AOL) configured to correct for aberrations and/or surface imperfections of the cornea of the eye.

A twenty-fourth (24^{rd}) object of the present invention is to provide an artificial ocular lens (AOL) configured to correct for aberrations and/or surface imperfections of the natural lens and cornea of the eye.

A twenty-fifth (25^{rd}) object of the present invention is to provide an artificial ocular lens (AOL) configured to correct for aberrations and/or surface imperfections of a prior implanted intraocular lens (IOL).

A twenty-sixth (26^{rd}) object of the present invention is to provide an artificial ocular lens (AOL) configured to correct for aberrations and/or surface imperfections of a prior implanted refractive correction lens (RCL).

A twenty-seventh (27^{rd}) object of the present invention is to provide an artificial ocular lens (AOL) configured to correct for aberrations and/or surface imperfections of a prior implanted intraocular lens (IOL) and refractive correction lens (RCL).

A twenty-eight (28^{th}) object of the present invention is to provide an artificial ocular lens (AOL) configured to visually correct a patient's eye for power, astigmatism and aberrations.

A twenty-ninth (29^{th}) object of the present invention is to provide an intraocular lens (IOL) configured to visually correct a patient's eye for power, astigmatism and aberrations.

A thirtieth (30^{th}) object of the present invention is to provide a refractive correction lens (RCL) configured to visually correct a patient's eye for power, astigmatism and aberrations.

A thirty-first (31^{th}) object of the present invention is to provide an anterior chamber refractive correction lens (acRCL) configured to visually correct a patient's eye for power, astigmatism and aberrations.

The present invention is directed to an artificial ocular lens (AOL), for example, an intraocular lens (IOL) (i.e. implantable artificial ocular lens (AOL) or implant for replacement of the natural eye lens) and a refractive correction lens (RCL) (i.e. implantable artificial ocular lens (AOL) or implant for refractive correction of the natural lens or a prior implanted intraocular lens (IOL) and/or a prior implanted refractive correction lens (RCL)).

A preferred embodiment of the artificial ocular lens (AOL) according to the present invention is a foldable or deformable phakic refractive correction lens (pRCL) (i.e. refractive lens added to to eye having a substantially healthy natural crystalline lens) or aphakic supplemental refractive correction lens (ap-sRCL) (i.e. refractive lens added to an eye having a replacement IOL lens) configured to be implanted through a very small incision in the eye (i.e. less than 2 mm and preferably 1 mm). A more preferred embodiment is a foldable or deformable anterior chamber phakic refractive correction lens (ac-pRCL) or aphakic supplemental refractive correction lens (ap-sRCL) configured to be implanted through a very small incision in the eye. A most preferred embodiment is a foldable or deformable custom anterior chamber phakic refractive correction lens (c-ac-pRCL) or aphakic supplemental refractive correction lens (ap-sRCL) configured to be implanted through a very small incision in the eye.

The intraocular lens (IOL) and refractive correction lens (RCL) according to the present invention preferably correct at least two, and more preferably at least three visual problems or defects (e.g. refractive problems, tissue problems, impairments, abnormalities, disease or other factors or conditions impairing or negatively affecting a patient's vision). In a preferred embodiment of the artificial ocular lens (AOL) according to the present invention, a patient's vision is corrected to 20:20, more preferably to 20:10, and even possibly to 20:7 and/or best correctable vision. In a most preferred embodiment, the artificial ocular lens (AOL) according to the present invention visually or optically corrects, protects, or otherwise overcomes any and all visual problems or defects.

The customized anterior chamber intraocular lens according to the present invention is manufactured or designed after thoroughly examining, measuring and mapping the patient's eye or eye vision. This information is compiled and then processed to custom manufacture or make the artificial ocular lens (AOL), in particular an anterior chamber phakic refractive correction lens (ac-pRCL) for the particular patient. For example, the patient's eye is evaluated for power correction, astigmatism correction, abnormal surface correction, abnormal refractive correction, abnormal tissue correction, and disease correction. For example, abnormal surface profiles or blemishes of the front and/or back surface of the

cornea and lens (e.g. natural lens or IOL) are analyzed by wavefront mapping of the vision of the eye, measuring the internal dimensions of the eye, including cornea, anterior chamber, iris, pupil, posterior chamber, capsular bag, retina, to determine the condition of the eye.

The information from the eye examination, measurements and mapping are processed through a mathematical formula or algorithm embodied in a computer program to calculate the biological, chemical, and physical parameters or characteristics of the artificial ocular lens (AOL) to be manufactured or made. Specifically, the exact lens size, lens thickness, lens length, lens width, optic location, optic shape, material, material physical properties, material chemistry, material surface chemistry, material refractive index, material hardness, material resilience, material elasticity, material finish, front lens surface conformation, back lens surface conformation, lens curvature, and other processing factors or parameters are determined, and then transformed into machine language for controlling highly precise and accurate computer operated manufacturing equipment (e.g. digitally operated tools) such as lathes, mills, grinding machinery, laser, surface finishing machinery, or any other type of machinery or processes that can be computer operated and controlled.

A preferred embodiment of the anterior chamber phakic refractive correction lens (ac-pRCL) according to the present invention adjusts the overall or macro power of the eye and corrects the astigmatism of the eye. Specifically, the lens optic is provided with 1) a lens optic for changing the overall or macro power of the eye; and 2) a lens optic for correction astigmatism of the eye. For example, the power correction of the lens optic can be obtained by cutting or contouring the main overall or macro shape and thickness of the lens optic and/or the lens optic can be multi-focal. The lens optic can be multi-focal by providing one or both surfaces of the lens optic with a multi-focal surface(s). The astigmatic correction of the lens optic can be obtained by providing a toric lens optic. For example, one or both surfaces of the lens optic to be toric surfaces.

A more preferred embodiment of the anterior chamber phakic refractive correction lens (ac-pRCL) according to the present invention adjusts the power of the eye, corrects the astigmatism of the eye, and corrects the fine or micro optics of eye based on wavefront analysis and mapping of the eye. For example, the power correction of the lens optic can be obtained by cutting or contouring the main overall or macro shape and thickness of the lens optic and/or the lens optic can be multi-focal and/or diffractive. The astigmatic correction of the lens optic can be obtained by providing a toric and/or diffractive lens optic. For example, one or both surfaces of the lens optic can be toric surfaces. Further; the lens optic can be made to provide point-to-point optical modification, adjustment, change or fine tuning of the structure and/or shape of the lens optic throughout the three dimensions (3-D) of the optical lens to micro fine tune or make micro modifications, micro adjustments or micro changes to lens optic on a micro basis to eliminate any and all optical aberrations and provide for full wavefront optical corrections.

A most preferred embodiment of the anterior chamber refractive correction lens (ac-pRCL) according to the present invention includes macro power adjustment, micro power adjustment, multi-focal, toric, and wavefront optics adjustment or correction on one or both sides of the lens optic, and/or within the interior of the lens optic.

The anterior chamber phakic refractive corrections lens (pRCL) according to the present invention is preferably custom made to correct any and all vision or optical problems or defects of the eye, including power correction, astigmatism correction, corneal surface and interior aberrations, lens surface and interior aberrations (natural or replacement lens, IOL), and other optical aberrations from other eye structure, eye aqueous and/or eye vitreous. In order to provided a custom anterior chamber phakic refractive correction lens (pRCL), it is required that the vision or optical defects of the eye are carefully measured, for example, by a visual field analyzer, slit lamp, biomicrosope and opthalmoscope. The goal is to provide an accurate and precise “eye assessment” to correct macro vision or optical defects or problems, and micro vision or optical defects or problems such as higher-order aberrations. The wavefront analysis based on adaptic measures of light deviations and aberrations can be measured to 0.01 microns (μm) equivalent to approximately 0.001 diopter (D) adjustment by root mean square deviations (RMS units). Standard refraction methods are used to measure macro visual or optical defects or problems such as low-order aberrations (second-order sphere or defocus and cylinder in 0.25 diopter (D) steps. Up to twenty percent (20%) of the higher-order aberrations come from the corneal, aqueous, lens, and/or vitreous accounting for numerous changes in the indices of refraction of light rays moving through the eye.

The higher-order aberrations require measuring equipment exceeding standard or conventional refractive measuring instruments. The higher-order aberrations include coma (third-order), trefoil (third-order), spherical aberrations and quadra foil (fourth-order), and irregular astigmatism (fifth-order to eighth-order). These higher-order aberrations provide refractive abnormalities well below 0.25 diopter (D unit) translating to three microns (μm) of tissue change within the eye. The wavefront analysis and mapping desired utilizes adaptive optics for measuring root mean square deviation (RMS) using measuring sensors such as a deformable “lenslent” systems to calculate RMS coefficients. The RMS coefficients are then converted into a polynomial pyramid (e.g. Zernike Pyramid). The three dimensional (3-D) models or two dimensional (2-D) color maps indicate lower and higher order aberrations of the eye. The Zernike polynomial measure aberrations up to the eleventh (11th) order, and can virtually analyze a hundred percent (100%) of the aberrations of the eye. Above the sixth (6th) order, only noise is created. Point spread functions (PSF) are used to measure and assess higher-orders aberrations in the human vision. These higher-order aberrations include distortions, haloes, tails, and/or double (overlapping) images.

The anterior chamber phakic refractive correction lens (ac-pRCL) can be made by selecting a material capable of being machined, and then cutting or contouring the front and back surface of the lens from a blank using a digital lathe, digital mill, laser, or by use of microlithography to form or make lens structure or markings. For materials that can be molded, the lens can be made by machining and polishing a mold cavity, and then molding the lens from a desired material. In a preferred embodiment, the lens mold utilizes a replaceable insert, in particular a replaceable molding pin for molding the lens optic portion of the lens. In this manner, the molding pin can be replaced each time a lens is molded to make a one of a kind custom lens optic for a particular patient. The remaining portions of the mold (e.g. to mold plate haptic portion) can be of a standard size and shape, and otherwise not customized.

The molding pin for molding the lens optic portion of the lens can be made by machining the molding pin surface thereof, and then highly polishing the molding pin surface. In a more preferred embodiment, the surface of the molding pin is machined, and then treated to provide a thin metal oxide layer thermally and/or electromagnetically deposited (e.g. vacuum deposited) to eliminate the need for the step of polishing the surface. Specifically, the molding pin is made of a copper/nickel alloy and the molding surface is diamond machined, and then a layer of corundum or aluminum oxide (e.g. sapphire, ruby, diamond (carboneaous)) is vacuum deposited on the molding surface to increase smoothness and durability thereof. The layer is preferably in the thickness range of fifty (50) to four-hundred (400) angstroms (Å).

A preferred embodiment of a phakic refractive correction lens (pRCL) according to the present invention includes two (2) separate pieces or parts, including a lens optic and a lens haptic. Preferably, the lens haptic is “V” shaped, and features two (2) relatively more rigid haptic arms formed of relatively higher modulus (harder) material(s), which haptic arms are flexibly resilient when thin. The lens haptic may also comprise less rigid haptic arms formed of relatively lower modulus (softer) materials bridging a discontinuity separating the haptic arms. The discontinuity may be coated with a lower modulus coating. The “V” shaped lens haptic allows for insertion of the lens haptic through a small incision in the eye, as small as about one millimeter (1 mm), without deforming the thin haptic frame. The lens haptic also features a fastening structure for fastening with the separate lens optic, preferably a fastening cleat. The foldable or deformable lens optic is then inserted into the eye through the same small incision (i.e. 2 mm or less), or more preferably ultra small incision (i.e. 1 mm or less), and attached to the lens haptic by the fastening cleat, by way of an aperture or eyelet provided in or on the lens optic.

The higher modulus resilient polymeric material may be selected from polyimide, polyetheretherketone, polycarbonate, polymethlypentene, polymethylmethmethl methacrylate, polypropylene, polyvinylidene fluoride, polysulfone, and polyether sulfone. Preferably, the higher modulus material is polyphenylsulfone (PPSU) or polyester or modified polyester such as liquid crystal polymer (LCP) liquid that can be molded into as thin or narrow as about 0.05 to 0.25 millimeters (mm). Preferably, the higher modulus material has a modulus of elasticity of about 100,000 to about 500,000 psi, even more preferably about 340,000 psi and has a hardness of about 60 to 95 on the shore D hardness scale, but more specifically a Rockwell R hardness of 120 to 130. The lower modulus resilient material may be an elastomer selected from silicones, urethanes, or hydrophobic or hydrophilic acrylics. Preferably, the lower modulus elastomeric material has a modulus of about 100 to 1000 psi (unit load at 300% elongation). Preferably, the lower modulus material has a hardness of about 15 to 70 on the shore A hardness scale. Preferably, the lower modulus material is a dispersion or optical silicone such as NUSIL MED 6400, 6600, 6604, 6605, 6607, 6640, 6755 and 6820, Nusil Technology, Carpinteria, Calif., USA, or the like.

In one embodiment of the phakic refractive correction lens (pRCL) according to the present invention, the relatively more rigid haptic arms define a “V”-shaped haptic frame. The haptic frame having two (2) haptic arms can be formed from a single uniform piece of material. The haptic arms may include fastening cleats for attachment of the lens optic. The haptic arms may additionally contain a slot open on one side to form a hinge which is bendable at the slot. The haptic arms may alternatively be provided each with a groove to form a hinge which is bendable at the groove.

The lower modulus material may partially or completely cover the haptic legs, or just the hinge area. In one embodiment, the lower modulus material is extended beyond the tip of the haptic legs to produce a softer contact point for the eye tissue. The lower modulus material may be applied by first surface treating the higher modulus material, and then molding the lower modulus material onto the treated surface. Preferably, the surface treatment is a corona or plasma or acid etched or a combination of treatment and additionally a primer. Preferably, the molding performed by dip molding, cast molding, or injection molding. Primers such as Nusil Med (product #CF1135) may also be used singly or in combination.

The lens optic may be any type of lens optic. Preferably, the lens optic is a refractive correction lens optic, or an interference (diffractive) lens optic to provide a thin optic. The lens optic can be toric, aspheric, multi-element, positive or negative, or other variable power focusing lens optic.

The present invention is also direct to a method for making an artificial ocular lens (AOL) with a haptic, including the steps of forming a thin frame lens haptic, coating a location of the lens haptic, and then breaking the lens haptic at the location of the coating. Further, the present invention is directed to a method of mounting a refractive correction lens (RCL) in the anterior chamber of an eye, including the steps of supporting a lens optic on a thin plate lens haptic extending between the angle of the anterior chamber; and then bending the lens haptic at a preferential hinge line to reduce pressure against the angle of the anterior chamber.

The refractive correction lens according to the present invention can also be used to correct vision or optical defects or problems from prior surgical procedures and/or implants (e.g. after LASIK refractive correction of the cornea, after implantation of an IOL).

### BRIEF DESCRIPTION OF THE DRAWINGS

FIG. 1 is a diagrammatic side cross-sectional view showing the general physiology of the eye with a first preferred embodiment of the refractive correction lens (RCL) according to the present invention implanted therein.

FIG. 2A is a top planar view of the refractive correction lens (RCL) shown in FIG. 1 implanted in the eye.

FIG. 2B is a top planar view of a second (2^{nd}) preferred embodiment of the refractive correction lens (RCL) according to the present invention similar to the first preferred embodiment shown in FIG. 2A, except the positioning of one of the haptic cleats is different.

FIG. 2C is a side elevational view of the refractive correction lens (RCL) shown in FIGS. 1 and 2.

FIG. 3 is a top planar view of the lens haptic of the refractive correction lens (RCL) shown in FIGS. 1 and 2.

FIGS. 4A-C are broken away top sequential view of one of the lens optic ears provided with an eyelet of the lens optic being connected to a modified haptic cleats having gripping ears of the lens haptic.

FIGS. 5A-E are sequential top planar views of the refractive correction lens (RCL) shown in FIG. 2A with the lens haptic being inserted through a small incision into an eye. The arrows indicate the manner in which the lens haptic is moved to allow insertion of the lens haptic without deformation.

FIGS. 5F-H are sequential top planar views of the refractive correction lens (RCL) shown in FIG. 2A with the lens optic being inserted through a small incision into an eye. The arrows indicate the manner in which the lens optic is folded and then insert into and through the small incision in the eye with forcepts.

FIG. 6A is a top planar view of another preferred embodiment of the lens haptic according to the present invention having an “L” shape.

FIG. 6B is a top planar view of a further preferred embodiment of the lens haptic according to the present invention having an “F” shape.

FIG. 6C is a top planar view of an even further preferred embodiment of the lens haptic according to the present invention having a “V” shape.

FIG. 7 is a top planar view of a lens haptic of a third (3^{rd}) embodiment of the refractive correction lens (RCL) according to the present invention having flexible haptic hinge portions supporting three (3) soft feet.

FIG. 8A is a top planer view of a fourth (4^{th}) preferred embodiment of the refractive correction lens (RCL) according to the present invention having three (3) attachment points between the lens optic and lens haptic for further stabilizing the lens optic thereon.

FIG. 8B is a top planar view of a fifth (5^{th}) preferred embodiment of the refractive correction lens (RCL) according to the present invention, including a lens optic provided with lens optic cleats and a lens haptic provided with slots for fastening the lens optic to the lens haptic (inverse fastening arrangement verses embodiment shown in FIG. 2A).

FIG. 8C is a top planar view of a sixth (6^{th}) embodiment of the refractive correction lens (RCL) according to the present invention, including a lens optic with a single lens optic eyelet and a lens haptic with a single haptic cleat. The embodiment shown on the right includes a lens optic provided with an optional stabilizing ear.

FIG. 8D is a top planar view of a seventh (7^{th}) preferred embodiment of the refractive correction lens (RCL) according to the present invention, including a lens optic with an elongated and stretchable ear having an lens optic eyelet to allow the lens optic to be partially attached to the lens haptic prior to insertion into the eye, and then allowing the lens optic to be subsequently inserted into the eye while being partially and continuously attached to the lens haptic.

FIG. 8E is a top planar view of an eighth (8^{th}) preferred embodiment of the refractive correction lens (RCL) according to the present invention, including a lens optic with a single large lens optic eyelet and a lens haptic having a pair of slots for connecting the lens optic to the lens haptic.

FIG. 8F is a top planar view of a ninth (9^{th}) preferred embodiment of the refractive correction lens (RCL) according to the present invention, including an elongated or oval-shaped lens optic to facilitate insertion through a small incision in the eye.

FIG. 8G is a top planar view of a tenth (10^{th}) preferred embodiment of the refractive correction lens (RCL) according to the present invention, including a round lens haptic with a portion removed to decrease the width of the lens optic to facilitate insertion through a small incision in the eye.

FIG. 8H is a top planar view of a lens optic of an eleventh (11^{th}) preferred embodiment of the refractive correction lens (RCL) according to the present invention.

FIG. 81 is a top planar view of a twelvth (12^{th}) preferred embodiment of the refractive correction lens (RCL) according to the present invention, including a lens optic provided with three (3) lens optic eyelets and a lens haptic provided with three (3) corresponding haptic cleats.

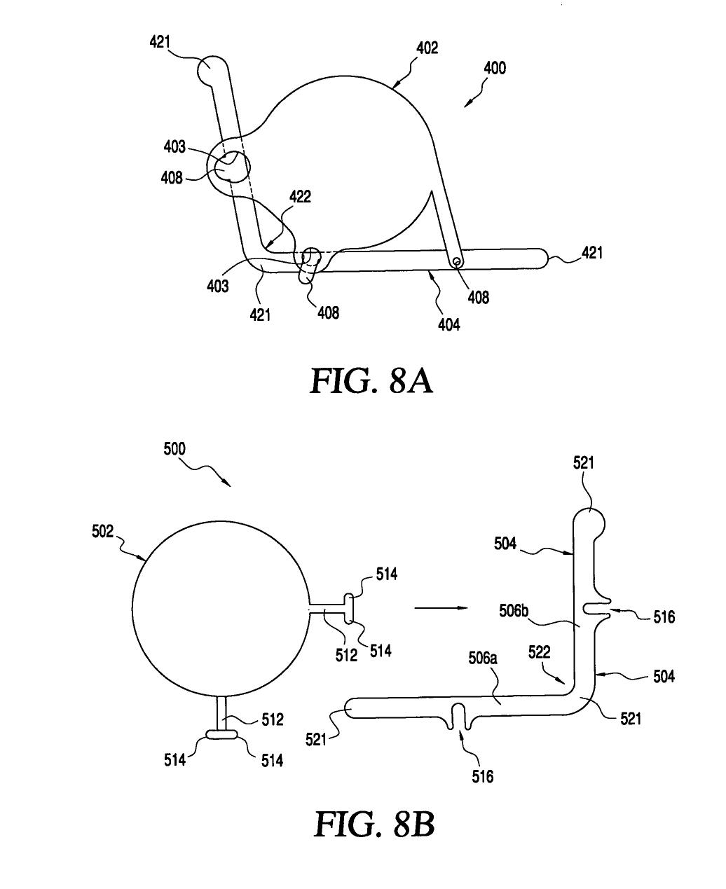

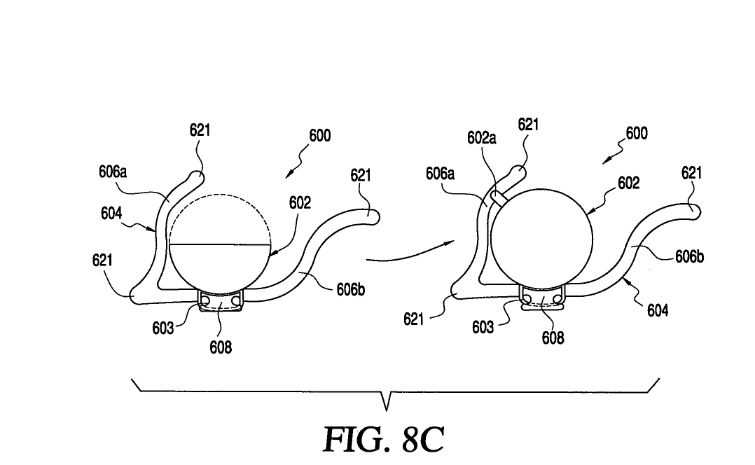

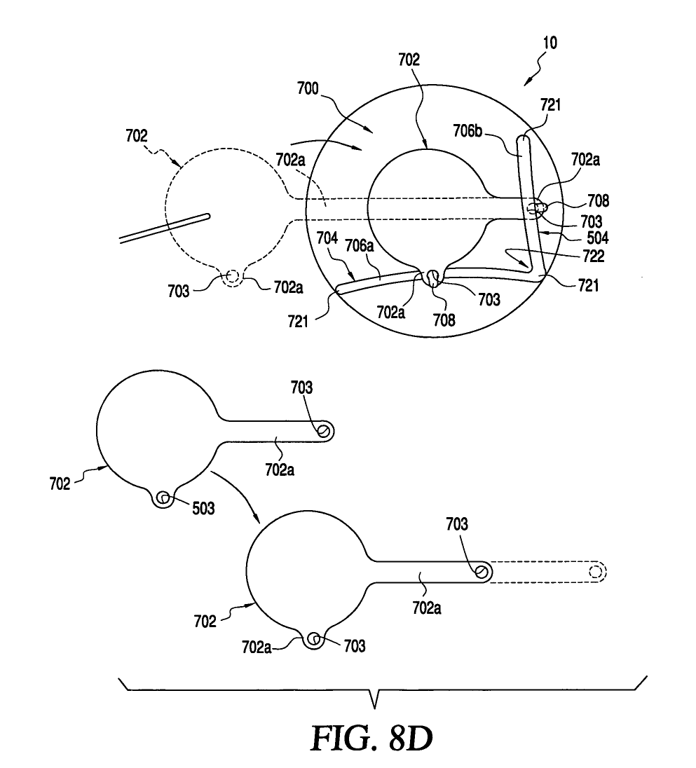

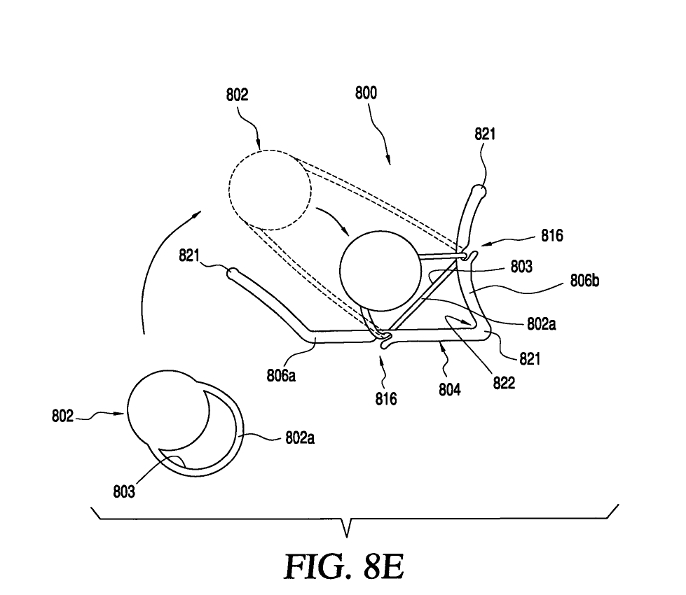

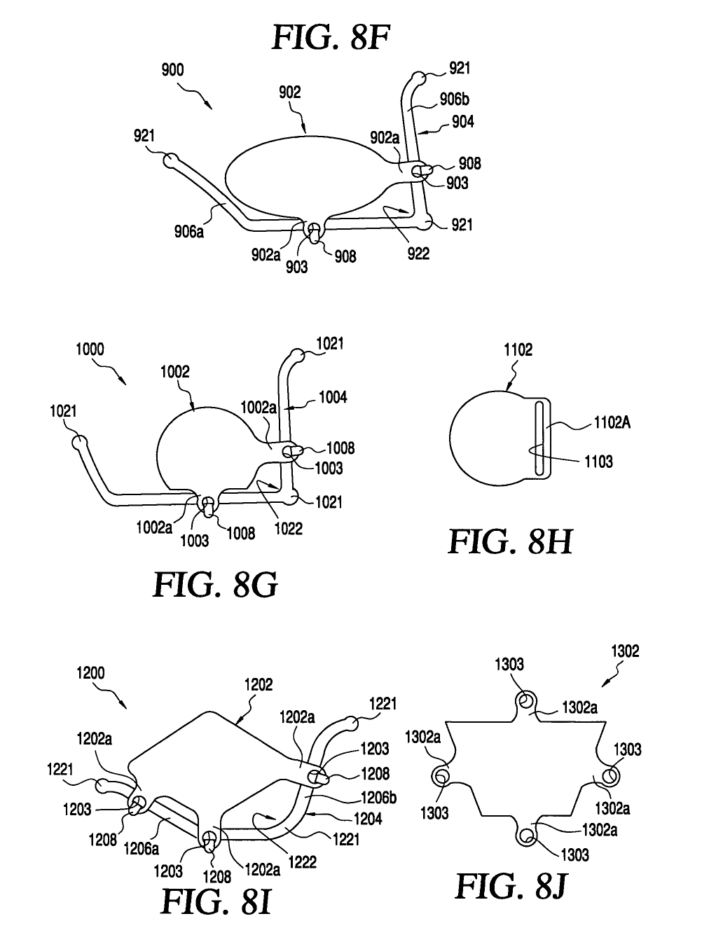

FIG. 8J is a top planar view of a thirteenth (13^{th}) preferred embodiment of the refractive correction lens (RCL) according to the present invention, including a lens optic provided with four (4) lens optic eyelets and a lens haptic provided with four (4) corresponding haptic cleats.

FIG. 10A is a top planar view of the front side of a lens optic of a preferred embodiment of the refractive correction lens (RCL) according to the present invention, including a multifocal.

FIG. 10B is a bottom planar view of the back side of the lens optic shown in FIG. 10A.

FIG. 11A is a top planar view of the front side of a lens optic of a preferred embodiment of the refractive correction lens (RCL) according to the present invention, including a multifocal.

FIG. 11B is a bottom planar view of the back side of the lens optic shown in FIG. 10A.

FIG. 12A is a top planar view of the front side of a lens optic of a preferred embodiment of the refractive correction lens (RCL) according to the present invention, including a multifocal.

FIG. 12B is a bottom planar view of the back side of the lens optic shown in FIG. 10A.

FIG. 13A is a top planar view of the front side of a lens optic of a preferred embodiment of the refractive correction lens (RCL) according to the present invention, including a multifocal.

FIG. 13B is a bottom planar view of the back side of the lens optic shown in FIG. 10A.

FIG. 14A is a top planar view of the front side of a lens optic of a preferred embodiment of the refractive correction lens (RCL) according to the present invention, including a multifocal.

FIG. 14B is a bottom planar view of the back side of the lens optic shown in FIG. 10A.

FIG. 15A is a top planar view of the front side of a lens optic of a preferred embodiment of the refractive correction lens (RCL) according to the present invention, including a multifocal.

FIG. 15B is a bottom planar view of the back side of the lens optic shown in FIG. 10A.

FIG. 16A is a top planar view of the front side of a lens optic of a preferred embodiment of the refractive correction lens (RCL) according to the present invention, including a multifocal.

FIG. 16B is a bottom planar view of the back side of the lens optic shown in FIG. 10A.

FIG. 17A is a top planar view of the front side of a lens optic of a preferred embodiment of the refractive correction lens (RCL) according to the present invention, including a multifocal.

FIG. 17B is a bottom planar view of the back side of the lens optic shown in FIG. 10A.

FIG. 18A is a top planar view of the front side of a lens optic of a preferred embodiment of the refractive correction lens (RCL) according to the present invention, including a multifocal.

FIG. 18B is a bottom planar view of the back side of the lens optic shown in FIG. 10A.

FIG. 19A is a top planar view of the front side of a lens optic of a preferred embodiment of the refractive correction lens (RCL) according to the present invention, including a multifocal.

FIG. 19B is a bottom planar view of the back side of the lens optic shown in FIG. 10A.

FIG. 20A is a top planar view of the front side of a lens optic of a preferred embodiment of the refractive correction lens (RCL) according to the present invention, including a multifocal.

FIG. 20B is a bottom planar view of the back side of the lens optic shown in FIG. 10A.

FIG. 20A is a top planar view of the front side of a lens optic of a preferred embodiment of the refractive correction lens (RCL) according to the present invention, including a multifocal.

FIG. 20B is a bottom planar view of the back side of the lens optic shown in FIG. 10A.

FIG. 21A is a top planar view of the front side of a lens optic of a preferred embodiment of the refractive correction lens (RCL) according to the present invention, including a multifocal.

FIG. 21B is a bottom planar view of the back side of the lens optic shown in FIG. 10A.

FIG. 22A is a top planar view of the front side of a lens optic of a preferred embodiment of the refractive correction lens (RCL) according to the present invention, including a multifocal.

FIG. 22B is a bottom planar view of the back side of the lens optic shown in FIG. 10A.

FIG. 23A is a top planar view of the front side of a lens optic of a preferred embodiment of the refractive correction lens (RCL) according to the present invention, including a multifocal.

FIG. 23B is a bottom planar view of the back side of the lens optic shown in FIG. 10A.

FIG. 24A is a top planar view of the front side of a lens optic of a preferred embodiment of the refractive correction lens (RCL) according to the present invention, including a multifocal.

FIG. 24B is a bottom planar view of the back side of the lens optic shown in FIG. 10A.

FIG. 25A is a top planar view of the front side of a lens optic of a preferred embodiment of the refractive correction lens (RCL) according to the present invention, including a multifocal.

FIG. 25B is a bottom planar view of the back side of the lens optic shown in FIG. 10A.

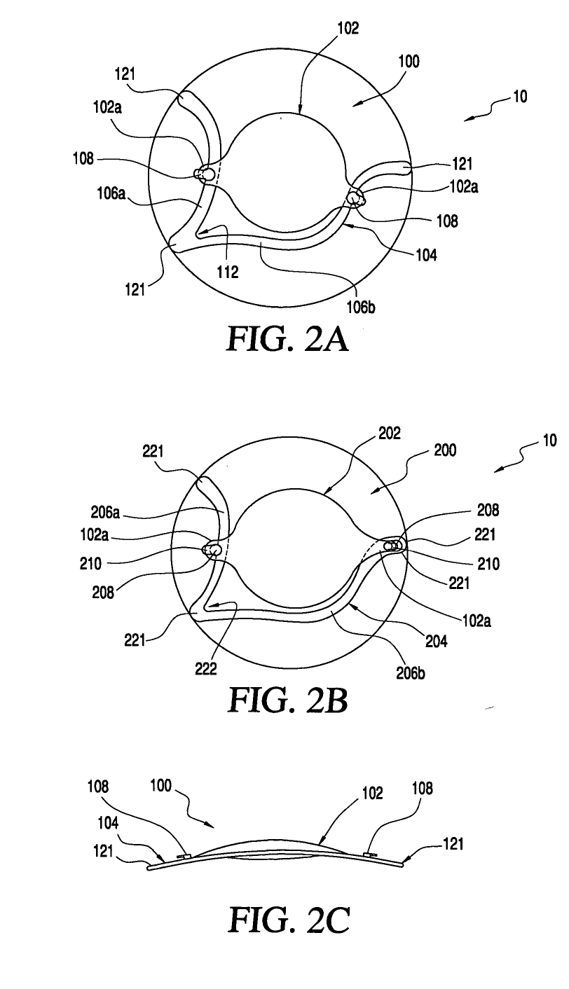

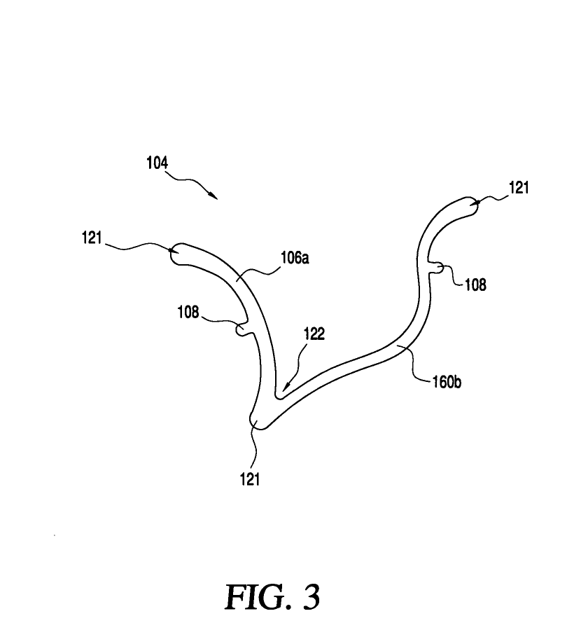

FIG. 26 is a partial broken away side elevational view showing the details of the connection between the lens optic and lens haptic shown in FIG. 2A.

### DETAILED DESCRIPTION OF THE PREFERRED EMBODIMENTS

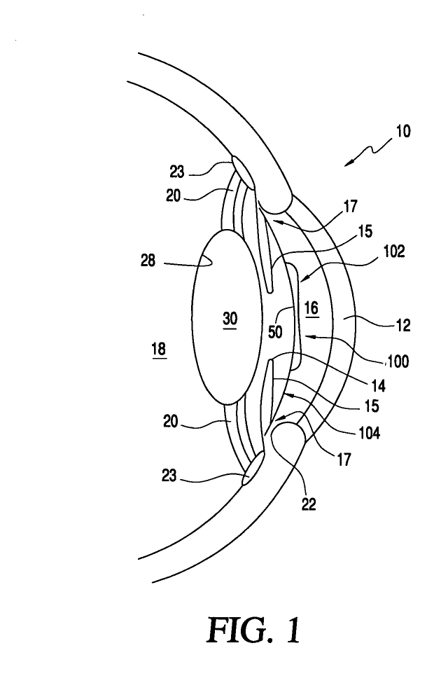

A preferred embodiment of a refractive correction lens (RCL) 100 according to the present invention is shown in FIGS. 1, 2A and 2C.

The refractive correction lens 100 is a two (2) part or piece lens, including a lens optic 102 connectable to a lens haptic 104. The lens optic 102 is made with a pair of lens optic ears 102a provided with lens optic eyelets 103. The lens haptic 104 has a “V” shape, and is preferably a thin film frame. The lens haptic 104 is insertable through a very small incision in the eye (i.e. one millimeter (1 mm) or smaller without deformation of the haptic). This lens haptic 104 is also lightweight, resilient, non-irritating, low cost, surgically implantable with a minimum of trauma to the eye, aesthetically pleasing, and does not support fibrous tissue growth in the anterior chamber 16 of the eye. The refractive correction lens 100 can be positioned in the anterior or posterior chamber of the eye as a phakic or aphakic lens. The lens haptic 104 includes a fastener for connecting to the separate piece lens optic 102. Preferably, the fastener is configured to allow the lens optic 102 to be assembled with the lens haptic 104 within the eye (e.g. anterior chamber).

The “V” shaped lens haptic 104 is a haptic system preferably made of a high modulus material. The lens haptic 104 may optionally be assembled with low modulus, soft, elastomeric and flexible hinge portions or zones. The more rigid frame of the lens haptic 104 in combination with the soft flexible hinges ensures that the lens optic 102 and lens haptic 104 assembly will maintain its shape and stay ideally situated or fixated in the angle 17 of the anterior chamber 16 of the eye, or in the posterior chamber depending on the application. While a lens haptic made of a soft material will not maintain a desirable shape and cannot properly support the lens optic from movement, the optional flexible hinge arrangement can automatically adjust to the normal movements of an eye.

As shown in FIG. 1, the front of the eye 10 includes the cornea 12, which serves as a refracting medium for incoming light into the eye in addition to defining the anterior wall of the eye 10. The pupil 14 or opening through the iris 15 provides a variable size aperture, and both eye structures are located behind the cornea 12. The iris 15 divides the eye 10 into the anterior chamber 16 and posterior chamber 18. A capsular bag 28 contains the natural crystalline lens 30. The capsular bag 28 is connected by zonular fibers 20 to a muscle of the eye 10 surronding the capsular bag 28 and natural crystalline lens 30 known as the ciliary muscle 23.

The lens optic 102 of the refractive correction lens 100 includes a separate centrally located optical zone, and may be configured for implantation into either the anterior chamber 16 or posterior chamber 18, and again may be used for a phakic or aphakic procedure. The lens haptic 104 of the refractive correction lens 100 extends radially outward in the center transverse plane of the lens optic 102.

As shown in FIG. 2A, the lens haptic 104 includes two (2) haptic arms 106a, 106b connected together at one end and set at a haptic arm angle 122. The haptic arms 106a, 106b support three (3) spaced apart haptic feet 121. The haptic arms 106a, 106b are arranged in an approximately “L” shape and define at least one “corner” or haptic arm angle 122 located between the two (2) haptic arms 106a, 106b. The haptic arm angle 122 can be up to approximately one-hundred thirty-five degrees (135°) or more, but preferably is about a ninety degress (90°) or less, more preferably between 35° and 60°, and most preferably about 45°. This arrangement allows the lens haptic 104 to be inserted through a small or very small incision made in the eye without deformation of the lens haptic 104. The small incision is preferably 2 mm or less, and the very small incision is preferably 1 mm or less. The maximum dimension across the width of the haptic arms 106a, 106b, at all points, is preferably less than the width of the incision. However, it is understood that due to the fact that living tissue is very elastic and will yield a little, the incision in the eye can be be made smaller and then stretched to accommodated the lens haptic 104 without damage to the tissue. For example, it has been observed a 2.5 mm incision can be stretched as large as 3 mm, to allow passage of a 3 mm wide lens haptic 104. Additionally, the lens haptic 104 can be made as narrow as about 0.05 mm at one or more areas or points to aid in the manipulation thereof.

The lens optic 102 when connected onto the lens haptic 104 is placed under a slight tension by the lens haptic 104. Specifically, the lens haptic arms 106a and 106b are moved slightly towards each other during the step of connecting the lens optic 102, and then released to slightly spring bias the lens optic eyelets 103 apart. Alternatively, the spacing between the lens optic eyelets 103 is such that when the lens optic eyelets 103 are looped over or connected to the haptic cleats 108 of the lens haptic 104, the lens optic eyelets 103 are distorted from round to oval and remain slightly oval in shape after the connection is made due to the tension force applied by the lens haptic 104 onto the lens optic eyelets 103. This tension force is convey through the lens optic 102 maintaining the lens optic 102 slightly under tension and the lens haptic 104 slightly under compression between the haptic arms 106a and 106b to prevent the lens haptic 104 from moving relative to the lens haptic 104 or being disconnected therefrom during implantation and use in the eye 10.

As noted in FIGS. 2A and 26, the lens optic ear 102a bends over the haptic cleat 108 and a top surface of the lens haptic 104, when assembled, and then downwardly (e.g. at an angle to perpendicular) relative to the lens plane of the lens optic 102. The lens optic ear 102a is highly flexible and bends and conforms to the upper and edge surfaces of the lens haptic 104. In an alternative embodiment, the lens optic ear 102a is made to be at an angle or perpendicular to the lens plane of the lens optic 102, and does not have to bend or be bent during assembly with the lens haptic 104.

In other alternative embodiments, the lens optic 104 is made off center (e.g. along X axis and/or Y axis, i.e. decentered) during manufacturing thereof. Alternatively, the the lens optic 102 and lens optic 104 can be made to be adjustable on-center or off-center after implantation in the eye by using a surgical instrument, or by using a light or heat source internal or external to the eye (e.g. using laser light to deform lens hatpic arms 106a, 106b or changing the haptic arm angle 121 or shortening or lengthening one or both of the lens haptic arms 106a, 106b by using laser light to further cross-link or decross-link the polymer material of the lens haptic 104). In the embodiment shown in FIG. 2B, the lens optic 102′ is slightly off-axis by moving the location of one or both of the haptic cleats 108 closer or further from the center of the lens optic 102 or relative to the inner angle 17 of the anterior chamber 16.

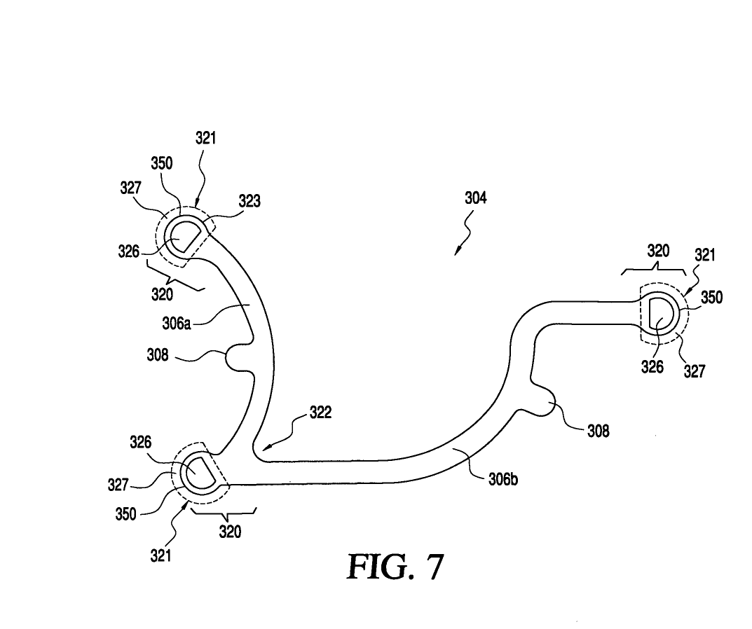

In a preferred embodiment shown in FIG. 7, the haptic feet 321 may be supported by flexible haptic hinge portions 320, which can be made in various manners, but have the property of being more flexible than the main portions of the haptic arms 306a, 306b. The flexible haptic hinge portions 320 are formed of a material that is more flexible and elastic than the rest of the lens haptic 304. In a more preferred embodiment, the flexible hinge portions 320 are covered by an elastomeric material layer 327, which extends from the haptic feet 321 around haptic toe portions 350. The flexible haptic hinge portion 320 can also be provided by making a thinner section of the haptic arms 306a, 306b, or providing a discontinuous opening in the haptic arms 306a, 306b where the elastomeric material layer 327 extends from the haptic feet 321 and around the haptic toes 350. The flexible haptic hinge portions 320 and haptic toe portions 350 can be produced in a variety of ways.

The lens optic 102 (FIG. 2) can be any type of lens optic. For example, the lens optic 102 can be made of elastomeric or polymeric optical material. The lens optic 102 can be a simple refractive lens optic, a mono-focal lens optic, toric or aspheric lens optic, a bifocal lens optic, a trifocal lens optic, a diffrative lens optic, an interference lens optic, a positive refractive lens optic or a negative refractive lens optic. The lens optic 102 can be made thinner by using a polychromatic diffractive lens arrangement such as disclosed in U.S. Pat. No. 5,589,982, which is hereby incorporated herein by reference. Optionally, the lens optic 102 can be made thinner by edge-bonding or bonding the haptic to the outside of the lens optic or drilling a hole into the side of the lens and anchoring one or more portions of the lens haptic 104 in a one (1) piece preassembled lens configuration.

As shown in FIG. 2C, the lens haptic 104 is curved or has a bow shape or is vaulted. The lens optic 102 can be made of silicone (e.g. optical index N=1.40 to 1.46), soft acrylic (e.g. N=1.40 to 1.46), hydrophilic acrylic or polymethylmethacrylate (e.g. N=1.49) or polyphenylsulfone (e.g. N=1.67). Alternatively, the lens optic 102 may be made of the same material as the lens haptic 104, and can be made of a material as low as 15 Shore hardness on the A scale.

The lens optic 102 can be attached to the lens haptic 104 in a variety of ways. A preferred embodiment is shown in FIG. 2A, in which the lens optic 102 is provided with lens optic eyelets 103, which permit attachment of the lens optic 102 to the pair of haptic cleats 108 provided on the lens haptic 104.

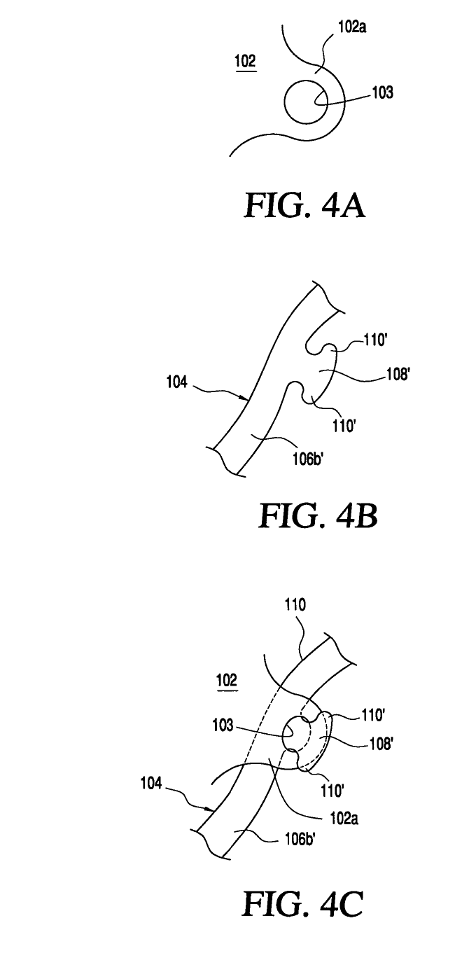

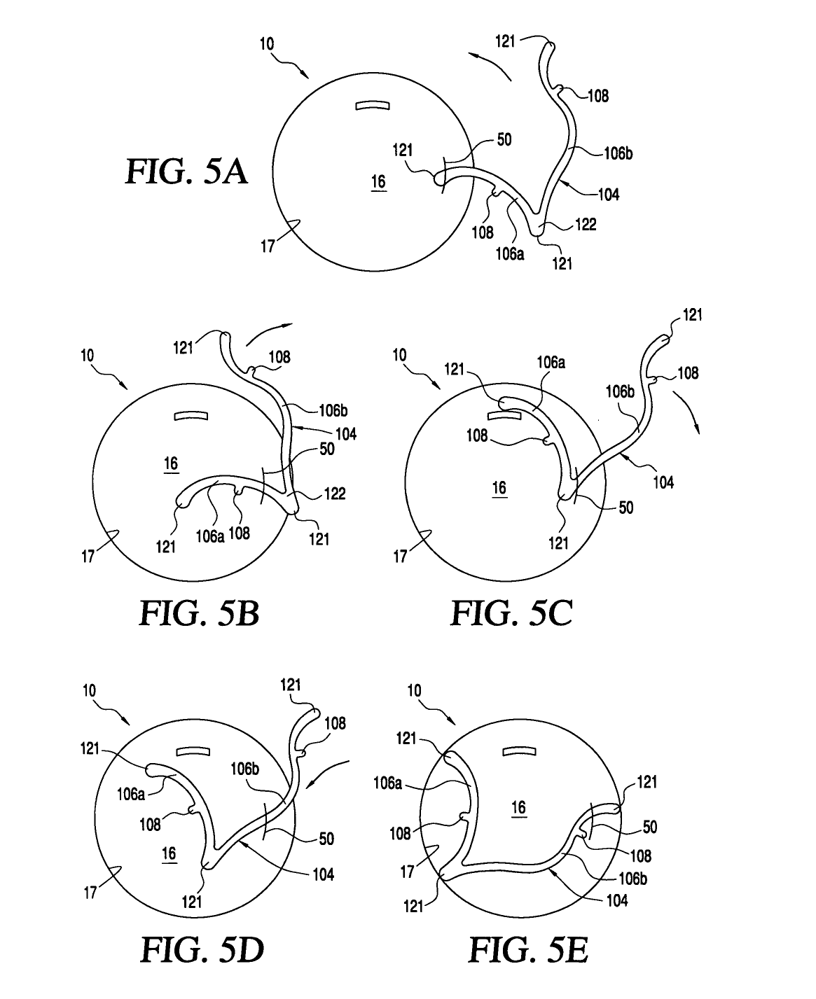

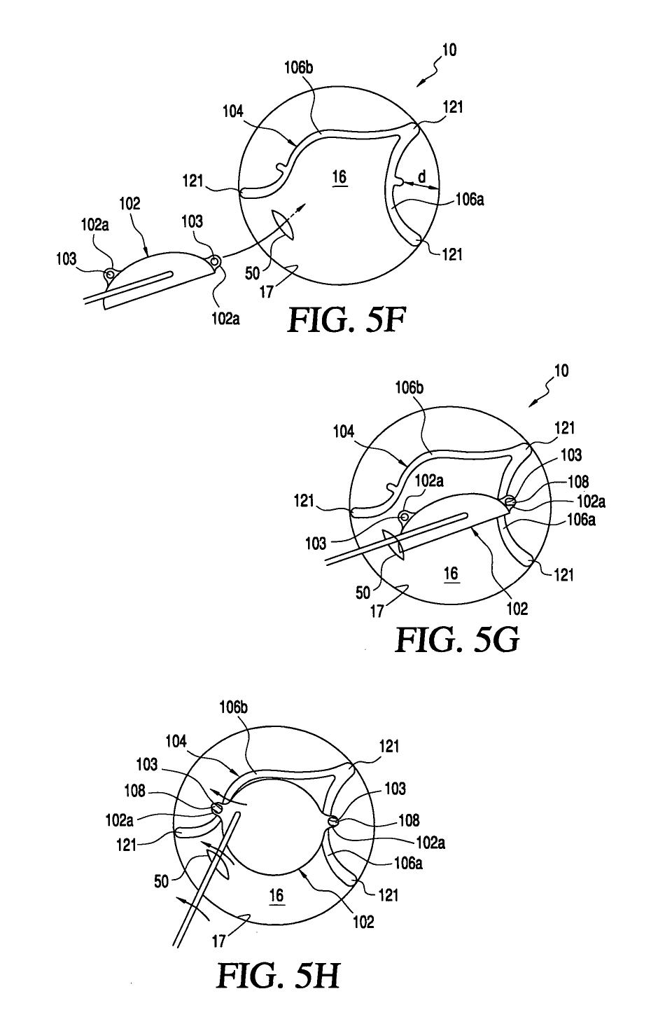

In a more preferred embodiment, as shown in FIGS. 4A-C, the haptic cleat 108′ is provided with haptic cleat ears 110′ shaped in such a manner to prevent the lens optic eyelets 103 from unfastening or disconnecting after assembly. The haptic cleats 108′ still allow the eye surgeon to attach the lens optic 102 to the lens haptic 104 within the eye using a forceps while providing improved anchoring. The lens haptic 104 is inserted through the very small incision through the eye, and then positioned in the eye as desired, as shown in FIG. 5A-E. Then, the lens optic 102 is rolled or folded, and then inserted through the small incision in the eye with forceps, and then attached to the furthest haptic cleat 108 from the opening (FIGS. 5F and G). As the forceps are removed, the optic eyelet 103 on the other side of the lens optic 102 can be attached to the lens haptic cleat 108 closest to the opening (FIG. 5H).

In a preferred embodiment, the lens optic 102 is produced of a material with a lower modulus than the lens haptic 104, thus allowing the lens eyelet 103 to be slightly stretched while the lens haptic 104 is rigid by slightly resilient to provide for a stronger attachment of the optic eyelets 103 to the haptic cleats 108 provided on the lens haptic 104. In one embodiment, one side of the lens optic 102 can be fastened to the lens haptic 104 before insertion of the refractive correction lens 100 into the eye. The lens optic 100 can be made with very thin edges (as thin as about 0.001 mm to reduce edge glare). The haptic cleats 108 again can be provided with prongs or ears to maintain the assembly of the optic eyelets 103 of the lens optic 102 on the haptic cleats 108 of the lens haptic 104 during use.

As shown in FIG. 2A, the haptic cleats 108 may be arranged such that they are not diametrically opposed. An advantage of this arrangement is that lens is not symmetrical to facilitate treatment of astigmatism. For example, if the refractive correction lens 100 needs to be inserted and positioned in a specific orientation, it can be more easily done with this asymmetry as a visual aid to the eye surgeon. In addition, multifocal lens optics can be used which allow for correction of a variety of eyesight imperfections. The addition of an optional third haptic cleat 408 (FIG. 8A) allows for control of asymmetric as well as symmetric features. The distance from the haptic cleat 108 (FIG. 2A) to the corneal angle 121 for the particular haptic cleat 108 should be more than the size of the lens optic eyelet 103 so that it can be easily fit onto the cleat.

The haptic cleats 300 are designed or configured to positively connect or securely fasten the lens optic 102 onto the lens haptic 104. This fastening system can be used to attach any type of refractive correction lens optic before insertion or after insertion into the eye. In addition, this arrangement will allow the eye surgeon a choice of lenses and lens powers to insert, and the surgeon can fasten one or more lenses onto the haptic cleats 108. Further, the haptic cleats 108 and/or the lens optic eyelets 103 can be tinted to further aid to the eye surgeon so as to be more visually identifiable to the eye surgeon during the implantation surgery.

The lens haptic 104 is preferably manufactured from a high modulus material. High modulus materials are generally relatively stiff, or hard, but resilient or springy, and permit relatively little bending before they break. Such materials are often brittle and have a high permanent set, but retain their shape after formation. Preferably, the high modulus material is a biocompatible thermoplastic film such as polyimide, polyester, polyetheretherketone, polycarbonate, polymethpentene, polymethymethyl methacrylate, polypropylene, polyvinylidene fluoride, polysulfone, polyether, and polyphenylsulfone. These are often referred to as “engineering plastics”. They have high tensile strength and are biocompatible, hydrolytically stable, and autoclavable for sterility, and have a high modulus ranging from a tensile modulus of about 100,000 to 500,000 psi (using test method D 638 of the ASTM). The material can be clear, opaque, or tinted, but is preferably clear. However, in many cases, even a tinted material if produced thinly enough, will appear clear in the eye. The lens haptic 104 can be made from a thin film sheet material cut by CNC machining, stamping, chemical etching, water jet cutting, and/or photo machining with an Eximer or YAG laser. The thin film sheet material may also be punched, stamped, perforated, photo-chemically or photo-optically shaped. An alternative method for production of the thin film frame lens haptic 104 includes molding the high modulus material into the desired shape. It is generally known in the plastics industry to identify thin film sheets of plastic material of less than 0.10 inches thick as “films”, and that definition is used herein. The lens optic eyelet 103 is provided with an aperture or hole of about 0.1 mm to 1.2 mm, preferably 0.5 mm in diameter. The thickness can be 0.001 to 0.010 inches, preferably 0.002 to 0.003 inches.

After cutting the shape of the lens haptic 104, the lens haptic 104 is shaped to be arcuate or vaulted by mounting the lens haptic 104 on a dihedral shaped tool or equivalent, and then baking the lens haptic 104 in an oven between 150° F. up to 550° F.

The thin film frame lens haptic 104 is typically then polished to remove any rough edges. The preferred method of polishing involves abrasive tumble or agitation polishing with glass beads. An alternative method for polishing the thin film frame lens haptic 104 and haptic feet 121 includes flame polishing. For embodiments of the phakic refractive lens provided with flexible haptic hinge portions 320 (FIG. 7), at least the areas of the thin film frame lens haptic 104 located away from the lens optic 102, which are to become hinges, are then treated so that an elastomeric compound or material can be attached thereto. An alternative surface treatment includes plasma surface treating (e.g. a low pressure corona treatment). Alternatively, the entire thin film frame lens haptic 104 can be surface treated or primed. Additionally, surface roughening such as by grit or vapor blasting can be used.

In a preferred embodiment of the refractive correction lens 100 according to the present invention, the lens haptic 104 is made of polyphenylsulfone material, which has a tensile modulus of about 340,000 psi (using test method D638 of the ASTM), is clear, and exhibits a natural UV light absorbance property below 400 nanometers (nm) resulting in a yellowish or amber tint. The thin film frame lens haptic 104 is preferably made from thin film, which is generally 0.025 cm (0.010 inches) thick, preferably about 0.001 to about 0.005 inches thick, or can be as thick as about 0.012 inches or as thin as 0.0005 inches. In a preferred embodiment, the haptic feet 121 are identical, but a non-identical haptic feet 121 configuration can be provided for use in an alternative embodiment depending on application. The thickness of the thin film frame lens haptic 104 contributes to its resilience or springiness, and lightness which is advantageous so that the refractive correction lens 100 is less likely to be disrupted from its initial positioning.

The refractive correction lens 100 is preferably thin and light in weight (e.g. approximately one-half (½) the weight of a standard lens, and can be between 2 to 10 milligrams and as low as 1 milligram in weight (in air) and 10% of this weight when in the aqueous of the eye. Preferably, the lens optic 102 is flexible, but may be made of a hard, stiff, low memory material. However, in the preferred embodiment, the lens optic 102 is made of silicone (e.g. a preferred silicone can be as low as 15 shore A hardness and the refractive index (N) value can be 1.430 to 1.460).

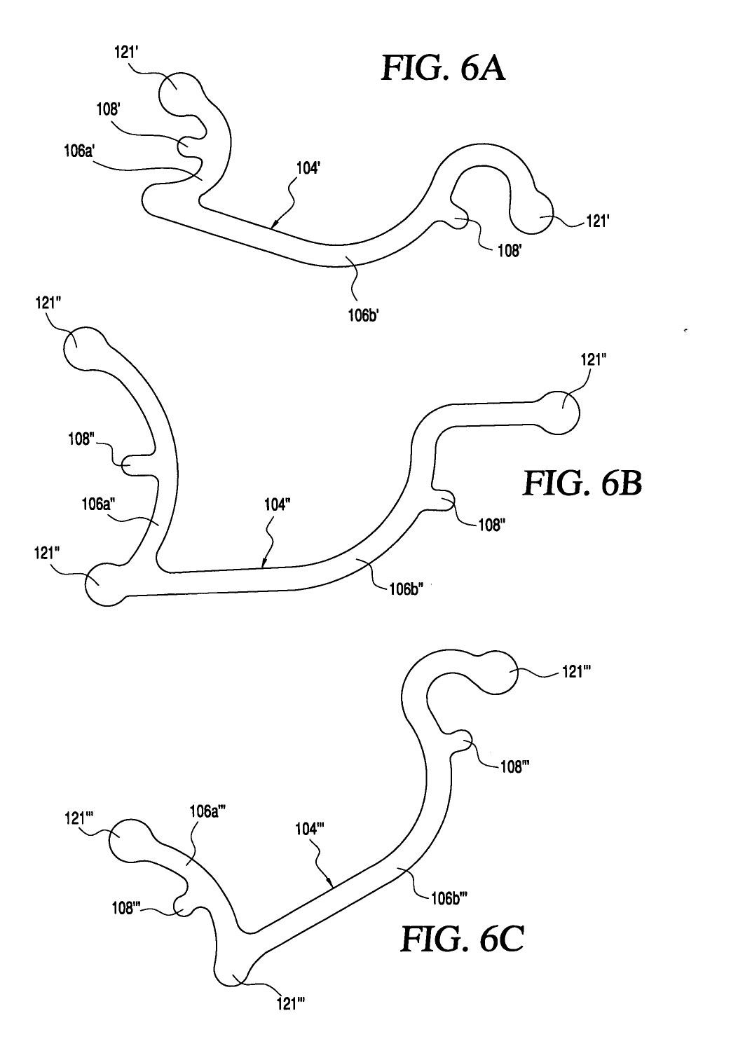

FIGS. 5A-E illustrate the sequence in which the lens haptic 104 can be inserted or manipulated through a very small incision 50 in the eye 10 without deformation. This substantially rigid lens haptic 104 arrangement is preferable a configuration which may possess a hinge or be “foldable” because it requires no lateral movement or unfolding within the very narrow confines of the anterior chamber 16 of the eye 10, which could contact the inner surfaces of the anterior chamber 16 and cause damage to the eye 10. In FIGS. 5A-E, the “L”-shaped lens haptic 104 allows for insertion through a very small incision 50 in the eye 10 by rotating the lens haptic 104 as it is inserted and manipulated within the eye 10. The dimensions of the lens haptic 104 preferably are such that the largest cross-sectional dimension at any point along the lens haptic 104 is less than 2 mm. FIG. 5A shows the lens haptic 104 initially being inserted into the eye incision starting at the shorter length haptic arm 106a of the L-shaped haptic up to the haptic angle 122 of the lens haptic 104 (FIG. 5B). At this point, the lens haptic 104 is manipulated such that the haptic angle 122 is inserted (FIG. 5C), and the lens haptic 104 is rotated until the shorter length haptic arm 106a of the lens haptic 104 lines up with the inner angle 17 of the anterior chamber 16 of the eye 10, and the longer length haptic arm 106b approximately perpendicular to the eye incision 50. The longer length haptic arm 106b is inserted by pushing the lens haptic 104 straight in (FIG. 5D). Because of the position of the incision 50 in the eye 10, the last step (FIG. 5E) may require a slight axial shortening of the lens haptic 104 by slightly resiliently biasing the longer length haptic arm 106b towards the fixed shorter length haptic arm 106a to fully insert the lens haptic 104 into the eye 10. Such spring biasing is distinguished from distortions, such as folding, bending, or rolling, normally used to introduce a deformable intraocular lens through a small incision in the eye. The “L” shaped lens haptic 104 shown, can be replaced with an “C” shaped, or “V” shaped lens haptics, as shown in FIGS. 6A and 6C.

After the lens haptic 104 is inserted through the small eye incision 50 and positioned within the eye 10 (See FIGS. 5A-E), the separate lens optic 102 is rolled or folded as required and inserted into the eye 10 with forceps, and attached to the furthest haptic cleat 108 from the small eye incision 50, as shown in the sequence of FIGS. 5F and 5G. As the forceps are removed, the lens optic eyelet 103 on the other side of the lens optic 102 can be attached to the haptic cleat 108 located closest to the small eye incision 50 (FIG. 5H).

As shown in FIG. 7, the lens haptic 304 includes three areas which come in contact with the interior eye tissue or inner angle 17 of the anterior chamber 16 of the eye 10. The haptic feet 321 and haptic toes 350 function like plate haptics, and as such, differ from fiber haptics. The hinged haptic toes 350 are attached to the haptic feet 121 in a manner so as to easily flexibly pivot and adjust to provide a better fit while maintaining lens centration.

The haptic feet 321 include haptic hinge portions 320. The haptic hinge portions 320 permit the haptic toe portions 350 to have a relaxed position, which can be at a slight angle to the plane of the thin film frame lens haptic 304 and the rest on the haptic feet 321. This slight angle permits each haptic foot 321 to fit into the anterior chamber 16 of the eye 10 in a manner so that the refractive correction lens 100 will be gently secured using low mechanical loads exerted by the flexible haptic hinge portions 120 combined with flexible haptic arms 106a and 106b. The flexible thin film frame haptic arms 106a, 106b can additionally be arcuately curved or shaped with a dihedral angle to more closely approximate the eye shape. More specifically, the haptic toe portions 350 are preferably made to define loops 323 (FIG. 7), such that one end of the loops 323 are spaced from the feet 321 and form an openings 326. The other ends of the loops 323 are attached to the feet 321.

As shown in FIG. 7, the flexible haptic hinge portions 320 are treated in such a way that a lower modulus material can be coated onto the higher modulus material completely, or partially, to connect the haptic toe portions 350 and haptic feet 321. The coating or layer for the haptic hinge portions 320 and haptic toe portions 350 is made from an elastomeric material which has a lower modulus (e.g. rubber) than that of the harder thin film frame lens haptic 104. A low modulus or softer material has high elongation and high memory to urge the haptic toe portion 350 back into its original position when compressed, and is preferably highly elastic. The more rigid thin film frame lens haptic 304 provides the conforming shape while the elastomer provides the resilient haptic hinge portions 320. The haptic hinge portions 320 connecting the haptic feet 321 to the rigid thin film frame lens haptic 304 functions, such that when bent, the outer elastic surface is placed under tension and the inner elastic surface is placed under compression. A variety of biocompatible elastomers such as urethanes and silicone dispersions such as NUSIL MED 6605, 6400, or 6820 and the like can be used as elastomers for covering the haptic hinge portions 320. The high modulus material can be surface treated using corona, plasma, or primers, individually or in combination. Next a primer is applied and lastly, the elastomer or low modulus material can be added by dipping the haptic feet 321 into the coating and subsequently curing it. The low modulus material is mechanically attached or chemically attached, and may be applied by cast molding as well as injection molding. In the preferred embodiment the process can be repeated. For example, the haptic hinge portions 320 and haptic feet 321 are dip coated multiple times with a dispersion, such dispersions containing solvents that evaporate leaving behind thinner coatings so that the thickness would be less than it would be if the dispersion were not in a solvent. However, alternative embodiments do not require multiple dipping. A protocol for the coating process is included in Example 1.

After coating, the haptic hinge portions 320 may be produced by breaking the high modulus material at the haptic hinge portions 320 by using scores or notches. This may be done by bending the haptic hinge portions 320 until the high modulus material hardens and breaks. Alternatively, the haptic hinge portions 320 may not need to be broken.

Alternative embodiments of the phakic refractive lens according to the present invention is shown in FIGS. 8A-H.

In FIG. 8A, a refractive correction lens 400 is shown which possesses three attachments. The refractive correction lens 200 possesses three (3) lens optic eyelets 403 of various sizes and shapes. The size of the angles between lens optic eyelets 403 of the lens optic 402 can be the same or different. This provides for angular non-symmetry. In the embodiment of the refractive correction lens 500 shown in FIG. 8B, the lens cleats 512 are provided on the lens optic 502 and slots, eyelets, apertures, or notches 516 are provided on the lens haptic 504. The lens optic 502 is attached to the lens haptic 504 by pulling the ears 514 of the lens cleats 512 through the slots 516.

In a further embodiment of the refractive correction lens 600 shown in FIG. 8C, one large haptic cleat 608 and one large lens optic eyelet 603 are used for connecting the lens optic 602 to the lens haptic 604. The lens 600 may have one or more additional tabs 602a for stability. In this embodiment, the lens optic 602 can be pre-attached and rolled for insertion with the lens haptic 604 much like the lens haptic 104 in FIGS. 5A-E, however, steps 5F-H would differ in that the lens optic 602 would simple “unroll” once the lens haptic 604 is in the correct position in the eye.

FIG. 8D shows another preferred embodiment of the refractive correction lens 700 in which the lens 700 is attached with a very stretchable eyelet 703 located at one attachment site, such that the lens haptic 704 can be inserted as shown in FIGS. 5A-E, with the lens optic 702 remaining outside of the incision. The lens optic eyelet 703 may elongate up to 300% of its length (see FIG. 8D). Then, as a last step, the lens optic 702 is rolled or folded, inserted through the small eye incision 50, and then allowed to be pulled or snapped back to its starting position on the lens haptic 704. The lens optic 702 can also include a second lens optic eyelet 703 to provide more stability to the lens optic 702 being fastened to the lens haptic 704. The lens optic eyelet 703 may alternatively have a sideways hole.

FIG. 8E shows a further preferred embodiment of the refractive correction lens 800 in which the lens optic 802 has a single large lens optic eyelet 803 which forms a stretchable band, and may be as wide as the lens optic 802. In this embodiment, the lens optic eyelet 803 can stretch away from the rigid lens haptic 804 during implantation of the lens optic 802. Once it springs back into position, a slight outward tension holds the lens optic 802 flat. In this case, the lens haptic 804 has two (2) notches 805 with which the lens optic eyelet 803 attaches at two (2) separate points on the lens haptic 804 to hold the lens optic 802 flat with a slight outward tension.

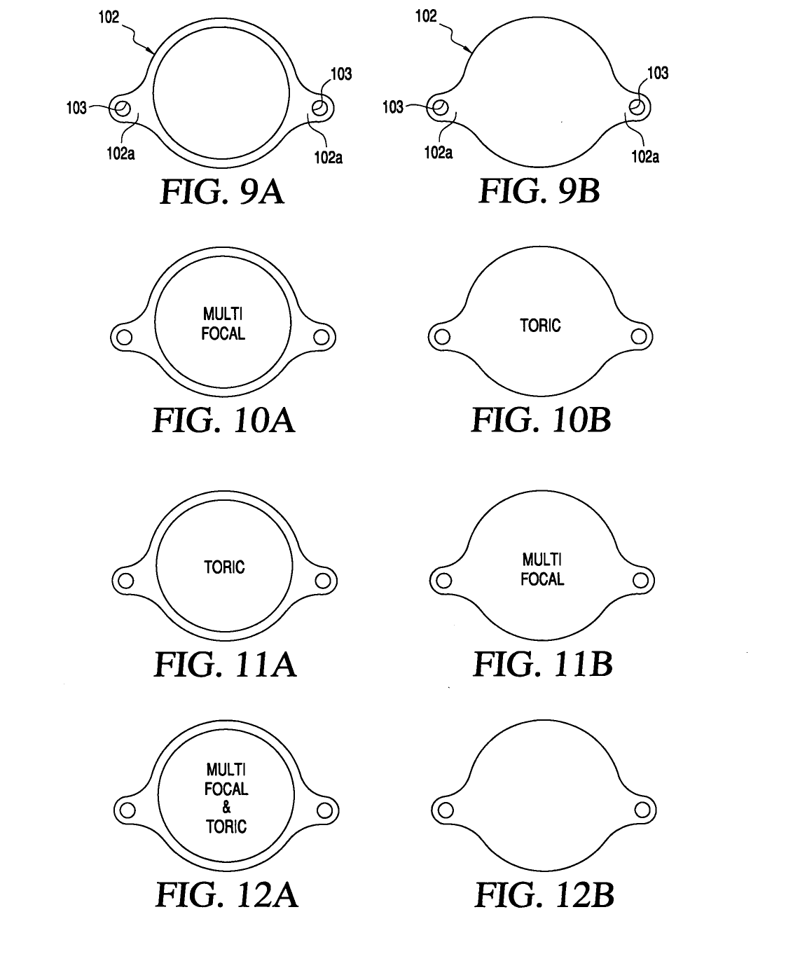

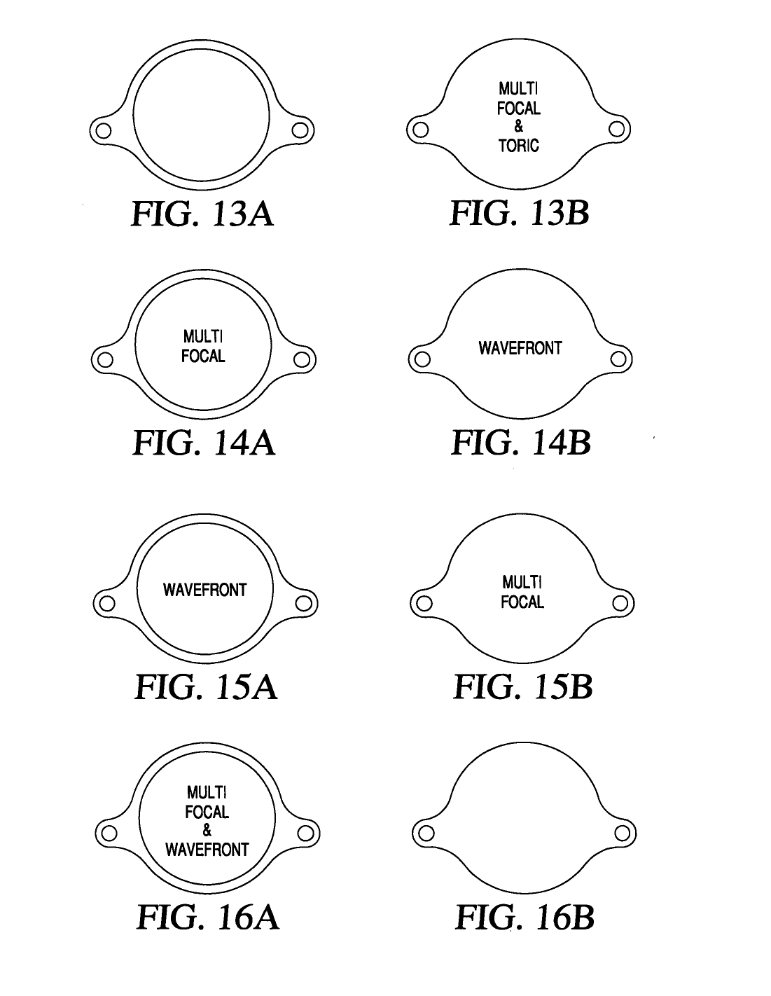

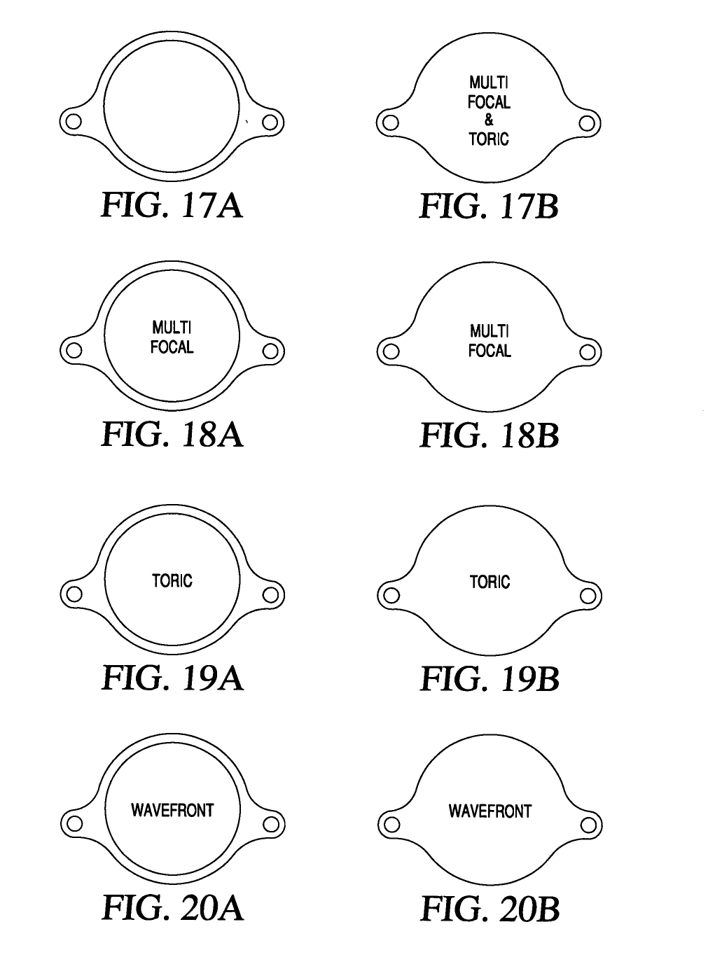

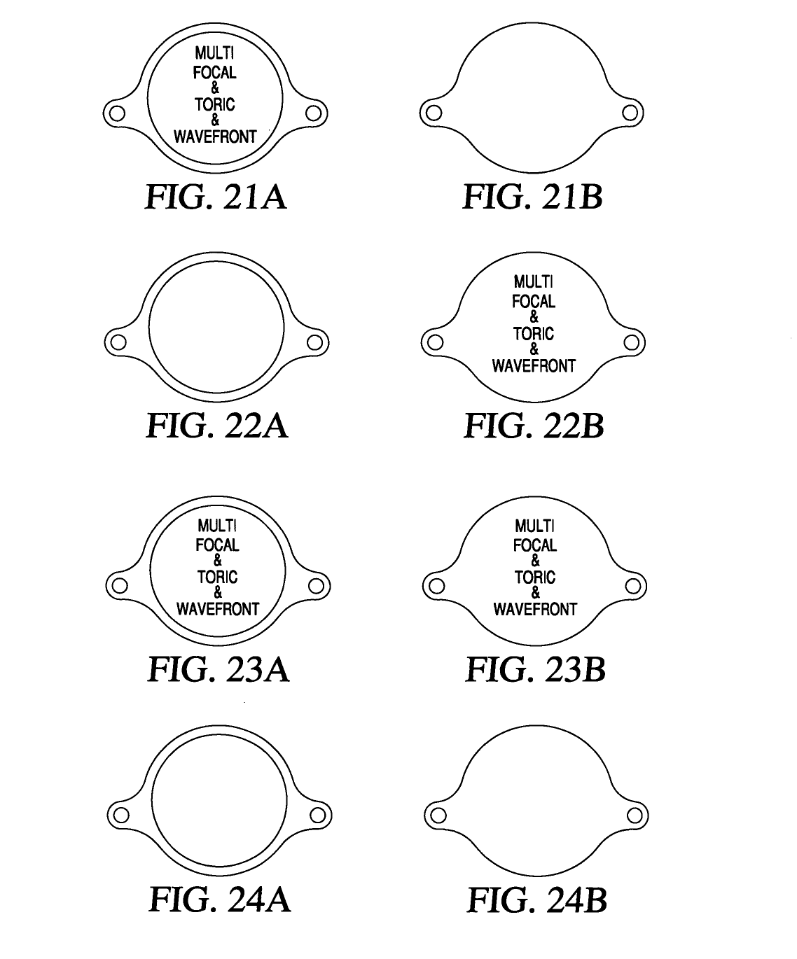

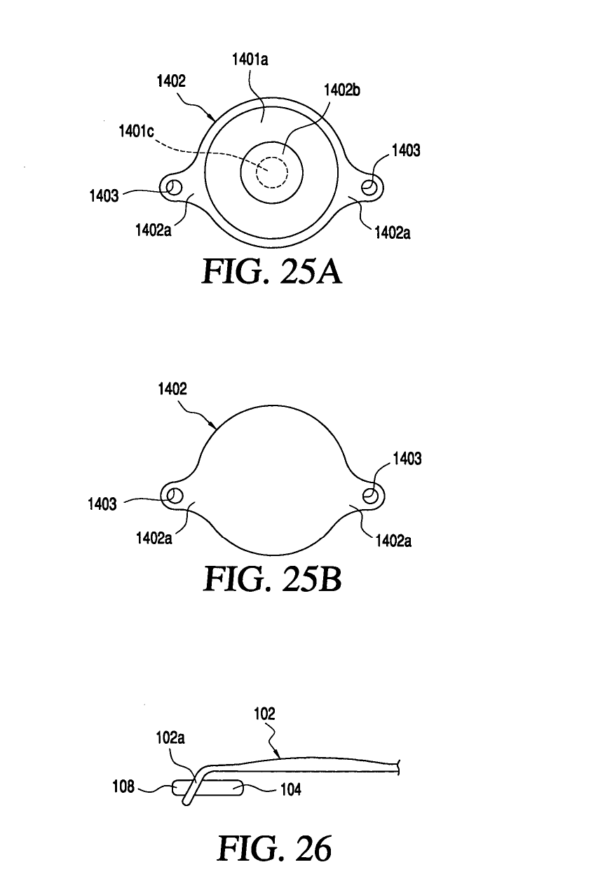

The lens optic 102 (FIG. 2A) can be circular shape, or oval shaped as shown in the refractive correction lens 900 in FIG. 8F, which advantageously makes the lens optic 902 narrower. In FIG. 8G, the refractive correction lens 1000 includes a lens optic 1002 that is segmented or chopped at one side to reduce the overall width of the lens optic 1002. In FIG. 8H, the refractive correction lens optic 1102 have an elongated slot shape eyelet 1003. In FIG. 81, the refractive correction lens 1200 includes a lens optic 1202 hving a parallelogram shape or even a trapezoid shape again allowing for a reduction in overall width. In FIG. 8J, the lens optic 1302 may have up to four (4) lens optic eyelets, or even up to six (6).

### EXAMPLE 1

### Insertion of the Two-Part Lens into the Eye

A two (2) mm small incision is made near the limbus of the eye. Buffers are injected into the anterior chamber of the eye 10. The lens haptic 104 is inserted as shown in FIGS. 5A-H by a rotation action. The eye surgeon grasps the folded lens optic 102 with the outside (distal) lens optic eyelet 103 leading forward. The eye surgeon then pushes the lens optic 102 through the small incision and hooks the distal lens optic eyelet 103 onto the distal haptic cleat 108 of the lens haptic 104. Then, the eye surgeon slowly opens the forceps while maintaining slight tension. The lens optic 104 is then grasped near or onto the closest lens optic eyelet 103 (proximal), and pulls it over the closer haptic cleat 108 of the lens haptic 104.

Therefore, the refractive correction lens 100 according to the present invention presents a number of advantages. It is inserted in two separate pieces significantly reducing the bulk so that the small incision can be as narrow as 1 mm. It is lightweight and reduces corneal chafing and pupilary block. In addition, because of the flexible haptic hinge portions 320 (FIG. 7) and haptic toes 350 are arcuate shaped, it is capable of being inserted and rest on the inner angle 17 of the anterior chamber 16e with a minimum of damage to the tissues as well as a minimum of discomfort to the patient. The plate haptic arrangement eliminates the problem of synechiae, and it can be used in a phakic or aphakic eye.

One advantage of the refractive correction lens 100 of the present invention is that the refractive correction lens 100 is a multi-part assembly and the characteristic or properties of each part of the refractive correction lens 100 can be retained. For example, the lens haptic 104 is rigidly and slightly resilient, and can be made to fit through a very narrow incision in the eye without deformation. The lens optic 102, preferably between 4 mm and 7 mm in diameter, can be inserted through the very small incision because it is constructed of a more pliable soft material that can be folded, squeezed or rolled, more so than it could be with the lens haptic 104 attached to be inserted into a considerably smaller incision. Therefore a multi-part refractive correction lens 100 according to the present invention allows for insertion into a much smaller incisions than an assembled lens.

The refractive correction lens 100 can be implanted into the eye 10 using a variety of surgical implant techniques known in the art. Although the preferred embodiment is a refractive correction lens 100 to be implanted into the anterior chamber 16 of the eye 10, using the inner angle 17 of the anterior chamber 16, the refractive correction lens 100 can also be implanted in the posterior chamber.

Additionally, any combination of the materials used will result in a refractive correction lens 100 that can be sterilized by a variety of standard methods such as ethylene oxide (ETO) or steam autoclaving at 250° F. or any other acceptable method and the lens will show long term biocompatibility and hydrolytic stability.

s 30 followed by implantation of an artificial lens involves a capsulorhexis incision in the capsular bag 28 that encloses the natural crystalline lens 30 located in the posterior chamber 18 of the eye 10 followed by phakoemulsification of the diseased natural crystalline lens 30 through a small incision 50 in the eye 10. The lens implant is then implanted back through the small incisiona and through the capsulorhexus into the capsular bag 28. For other types of procedures, the natural crystalline lens 30 is not removed, and a phakic refractive lens (PRL) is implanted in the anterior chamber 16 or in front of the natural lens 30 in the posterior chamber 18 of the eye 10.

The artificial ocular lens (AOL) according to the present invention can be an intraocular lens (IOL) designed or configured for replacement of the natural crystalline lens and/or a refractive correction lens (RCL) for refractive correction of the natural crystalline lens or refractive correction of a replacement lens or IOL.

The refractive correction lens according to the present invention is designed or configured to visually or optical correct multiple visual or optical problems of the eye.

The lens optic 102 is provided with a front side, shown in FIG. 9A and a back side shown in FIG. 9B. In this embodiment, the front and back surfaces of the lens optic 102 are provided with refractive surfaces for power correction of the eye.

A variety of different embodiments of the lens optic of the refractive correction lens (RCL) according to the present invention is shown in FIGS. 10A-B through FIG. 24A-B. The refractive corrections lens shown are provided with refractive surfaces on the front and/or back side of the lens optic.

In the embodiment shown in FIGS. 10A and 10B, the front surface of the lens optic is provided with a multifocal surface and the back side of the lens optic is provided with a toric surface.

In the embodiment shown in FIGS. 11A and 11B, the front side of the lens optic is provided with a toric surface and the back side of the lens optic is provided with a multifocal surface.

In the embodiment shown in FIGS. 12A and 12B, the front side of the lens optic is provided with a multifocal surface and the back side of the lens optic is provided with a toric surface.

In the embodiment shown in FIGS. 13A and 13B, the front side of the lens optic is provided with a refractive surface and the back side of the lens optic is provided with a multifocal and toric surfaces.

In the embodiment shown in FIGS. 14A and 14B, the front side of the lens optic is provided with a multifocal surface and the back side of the lens optic is provided with a wavefront surface.

In the embodiment shown in FIGS. 15A and 15B, the front side of the lens optic is provided with a wavefront surface and the back side of the lens optic is provided with a multifocal surface.

Diopter

Blue blocking up to 400 up to 450 up 500 up to 550 nanometer (50% transmisson of 450 nm)

Yellow

No uv inhibitor, or uv inhibitor

In the embodiment shown in FIGS. 16A and 16B, the front side of the lens optic is provided with a multifocal and wavefront surfaces and the back side of the lens optic is provided with a refractive surface.

In the embodiment shown in FIGS. 17A and 17B, the front side of the lens optic is provided with a refractive surface and the back side of the lens optic is provided with a multifocal and wavefront surfaces.

In the embodiment shown in FIGS. 18A and 18B, the front side of the lens optic is provided with a multifocal surface and the back side of the lens optic is provided with a multifocal surface.

In the embodiment shown in FIGS. 19A and 19B, the front side of the lens optic is provided with a toric surface and the back side of the lens optic is provided with a toric surface.

In the embodiment shown in FIGS. 20A and 20B, the front side of the lens optic is provided with a wavefront surface and the back side is provided with a wavefront surface.

In the embodiment shown in FIGS. 21A and 21B, the front side of the lens optic is provided with multifocal and toric and wavefront surfaces and the back side of the lens optic is provided with a refractive surface.

In the embodiment shown in FIGS. 22A and 22B, the front side of the lens optic is provided with a refractive surface and the back side of the lens optic is provided with a multifocal and toric and wavefront surfaces.

In the embodiment shown in FIGS. 23A and 23B, the front side of the lens optic is provided with at least two (2) of a multifocal surface, toric surface and/or wavefront surface and the back side of the lens optic is provided with at least two (2) of a multifocal surface, toric surface and wavefront surface.

In the embodiment shown in FIGS. 24A and 24B, the front side of the lens optic is provided with a multifocal and toric and wavefront surfaces and the back side of the lens optic is provided with multifocal and toric and wavefront surfaces.

In the embodiment shown in FIGS. 25A and 25B, the front side of the lens optic 1402 is provided with a two (2) multifocal and/or diffractive lens zones or surfaces, including a circular-shaped center multifocal and/or diffractive lens surface 1401a and a concentric outer ring-shaped multifocal and/or diffractive lens surface 1401b on the front surface thereof. Optionally, one multifocal and/or diffractive lens surface or zone can be provided on one (1) side of the lens optic 1402 and the other multifocal and/or diffractive lens zone can be provide on the opposite side. As a further option, multiple multifocal and/or diffractive lens surfaces or zones can be provided on both the front surface and back surface of the lens optic 1402.

For a presbyopic embodiment of the refractive correction lens 1402 according to the present invention, for example, the central additions for the lens surface 1401a should be +3.00 diopters (D). Similar but slightly different refractive correction lenses 1402 can be made for early presbyopes and late presbyopes. For example, for early presbyopes, lens surface 1401a should be +0.5 diopters (D) and for late presbyopes, lens surface 1401a should be +3.0 diopters (D). The central lens surface 1401a should be around 3 millimeters (3 mm).

As a further embodiment, the refractive correction lens 1402 can be provided with a third multifocal surface or zone 1401c to provide a trifocal (e.g. −1, 0, +1). For example, three (3) object distances, the type of structure (e.g. sine wave, trapezoid and/or rectangle), and the lens material can be specified for making the trifocal embodiment. In other embodiment, more than three (3) multifocal surfaces or zone (e.g. concentric, symmetric, asymmetric, matrix arrangements of surfaces or zones) can be used for particular applications or custom made for a particular eye. Alternatively, lithography can be used to print marks or a pattern on one or both surfaces of the lens (e.g. grid, rings, matrix) to cause light diffraction to make a diffractive lens optic, or lithography combined with etching (e.g. lens mold surface) can be used to make nanometer to angstrom dimension profiles, protrusions, patterns, contours on lens surfaces to provide multifocal and/or defractive lens surfaces.

To make the custom refractive correction lens (RCL) according to the present invention, the patient's eye must be carefully analyzed, measured and mapped to determine the specifications of the refractive correction lens (RCL) to be manufactured. Specifically, the following is a list of specifications of the refractive correction lens (RCL) to be considered and then specified:

| 1) | refraction | | Exact Diopter (D) to 0.00 D | |
|---|---|---|---|---|
| 2) | diffraction | | | |
| 3) | aspheric | | yes/no, any special degree | |
| 4) | presbyopia | | yes/no | |
| 5) | multifocal optic | | 50 cm to infinity | |
| | bifocal | | | |
| | tTrifocal | | | |
| | accommodating IOL | | 38 cm to infinity | |
| | combinations | | 19 cm to infinity | |
| | bifocal | | | |
| | trifocal | | | |
| 6) | astigitism | | | |
| | how much | | diaopters | |
| | where located | | degrees | |
| | what shape | | many | |
| 7) | Aberration | | | |
| | cornea | | | |
| | lens | | | |
| | retina | | | |
| | combined | | | |
| | what shape | | | |
| | where located | | | |
| | how much | | | |
| 8) | optic | | | |
| | size | | 2.5 to 7 mm | |
| | shape | | round elliptical other | |
| | location | | centered or decentered | |
| | where | | degrees | |
| | concentric | | yes/no | |
| | symmetrical | | yes/no | |
| 9) | overall lens size | | made to fit eye or bag | |
| | shape | round | 8 to 15 mm | |
| | | elliptical | 8 to 15 mm | |
| | | other | 8 to 15 mm | |
| 10) | material | silicone | clear | yellow |
| | | acrylic | clear | yellow |
| | | other | clear | yellow |

| | |
|---|---|
| blue light blocking | yellow is the blue light |
| | blocking mechanism |

| | | |
|---|---|---|
| 11) | transmission of date | eye model data, |
| | manufacturing IOL from data | topography-trace data |
| | testing IOL from data | |

### EXAMPLE 2

The following is an example of a patient information request form to gather information for prescribing and specifying a custom refractive correction lens (RCL) according to the present invention.

| 1) | Dr. Name | | | | | |
|---|---|---|---|---|---|---|
| 2) | Dr. Practice Name | | | | | |
| 3) | address | | | | | |
| 4) | phone number e-mail address | | | | | |
| 5) | patient Name | | | | | |
| 6) | patient Code | | | | | |
| 7) | which Eye | | | OS | OD | Both |
| 8) | AC Depth | | | | | |
| 9) | axial length | | | | | |
| 10) | refraction (Exact, 00D) | | | | | |
| 11) | aspheric correction Yes No/Amount | | | | | |
| 12) | presbyopia Yes/No | | | | | |
| | preferred reading distance | | | | | |
| | how close up? (19 cm to 50 cm) | | | | | |
| | which Lens Design | | | | | |
| | accommodating (38 CM) | | | | | |
| | multifocal (50 cm) defractive/refractive | | | | | |
| | tri-focal/bi focal | | | | | |
| | combination (19 cm to 50 cm) | | | | | |
| | trifocal/bifocal | | | | | |
| 13) | astigmatism, describe: | | | | | |
| | (amount) | | | | | |
| | (location in degree) | | | | | |
| | with rule against rule oblique | | | | | |
| | other-describe | | | | | |
| 14) | aberration: Best Zernike Model | | | | | |
| | cornea/lens/retina/total | | | | | |
| | amount | | | | | |
| | location | | | | | |
| | cornea | | | | | |
| | spherical aberration | | | | | |
| | high order astigmatism | | | | | |
| | trefoil | | | | | |
| | other describe | | | | | |
| 15) | other items needed | | | | | |
| | pupil concentric/non-concentric | | | | | |
| 16) | material | silicone | acrylic | collagen | polyimide | other |

| | |
|---|---|
| | preference |
| | Blue light blocking? |
| 17) | optical size 2.5 to 7 mm |
| | overall diameter 8 to 15 mm |
| 18) | optical symmetrical/non-symmetrical/excentric |

At the eye surgeon's office, the patient's eye is measured using visual field analyzers, eye charts and a topographer/abberometer. The abberometer measures the aberrations in the patient's eye and provides the eye surgeon with a topography map outlining all the aberrations. The eye surgeon uses the abberometer to check where the aberrations are coming from and analyze the data for different pathologies and make changes to the data where necessary. The abberometer is then used to generate a topography map and digital data that will be transferred to the manufacturer in the form of a customized lens order via satellite, internet, telephonic down load, CD ROM, DVD or mail or fax. Abberometer obtains the necessary information by using the Shack Hartman or means, which analyzes multiple beams of light transferred to the retina and then returned back through the eye. Variations of the light are measured against a light standard that would give perfect vision if all the parameters are met. The variation of the light is then compared against the Zerkeny polynomial to determine whether the variations are in the form of low order aberrations usually spherical and cylinder (toric) or high order aberrations such coma; trefoil (shapes showing in the optic system that look like a starburst usually around the periphery of the eye extending toward the center.

This information will then be received by the manufacturer, analyzed for completeness and any other kind of transmission errors. The data received is in the form of data points to be run through a program that to invert or reverse the information, since to correct an optic system requires making points or corrections that are opposite of the actual data received. This data will be run through the program to convert the data converted into machine language that will form a JFL file that will tell any equipment that can have varying cut (the Presitech Optiform with a variable forming tools or the DAC system with its toric generator) to cut a mold pin or optic in a form based on the information received from the eye surgeon's topography/aberrameter, Zydekia Chart etc.

The order depending on the method of manufacturing can create a lens optic as part of the shop order or create a mold pin for the shop order in case of silicone manufacturing. The shop order would then go through the manufacturing process for developing lenses and a final lens optic would be made. During the process the lens would be marked in a manner so that the eye surgeon doing the surgery can tell where on the lens optic the changes are made. One side of the optic can contain all the changes needed for a multifocal, toric and/or wavefront corrections, or some changes can be on the front side and some on the back side of the refractive correction lens (RCL) depending on the patient and manufacturing constraints. In order to know that what was manufactured is what was ordered, similar equipment would be used to generated the data such as an abberometer using the same theoretical method to measure the reverse aberrations created in the lens and compare it with the original input information. The refractive correction lens (RCL) is then sterilized and sent to the eye surgeon.

The manufactured lens data can be sent back with the lens to the eye surgeon, including data points and topography map with a manufacturing certificate for the eye surgeon and patient similar to a patient ID card, instead it would have a topography of the lens on the card.

The eye surgeon then inserts the lens haptic and lens optic into the patient's eye and places the refractive correction lens (RCL) where needed based on what was ordered received. Minor adjustments in the lens optic and lens haptic can be made to obtain the appropriate axis of the optic. It is possible to make and optic off center in a mold pin combination if it were determined up front exactly where and if the optic needed to be changed from its center point. It is also possible to put adjustments items on the optic and haptic whereby the optic could be shifted up, down or side ways so that the multifocal, toric, wave front can be lined up to give the patient better vision.

While this invention has been described with respect to various specific examples and embodiments, it is to be understood that the invention is not limited thereto and that it can be variously practiced within the scope of the following claims.

We claim:

1. An anterior chamber refractive correction lens device configured for implantation through a small incision in the eye, said device comprising:
a deformable lens optic configured to be inserted through the small incision in the eye, said lens optic being a refractive and multifocal lens optic; and
a substantially rigid lens haptic, said lens optic and said lens haptic being configured to be connected together within the anterior chamber of the eye to provide an assembled visually and optically functioning anterior chamber refractive correction lens device, said lens haptic placing said lens optic under slight tension when assembled together to prevent said lens optic from moving relative to said lens haptic and to prevent the inadvertent disconnection of the lens optic from the lens haptic during implantation and use in the eye.

2. An anterior chamber refractive correction lens device configured for implantation through a small incision in the eye, said device comprising:
a deformable anterior chamber lens optic configured to be inserted through the small incision in the eye into the anterior chamber of the eye and fit within the anterior chamber of the eye, said lens optic being a refractive, multifocal and toric lens optic;
an anterior chamber lens haptic configured to be inserted through the small incision in the eye into the anterior chamber of the eye and then fit within the anterior chamber of the eye, said lens haptic being sufficiently stiff and resilient to allow a portion of said haptic portion to be bowed without breaking, said lens haptic configured to connect with said lens haptic at two or more spaced apart locations and place a lens optic portion located between said two or more locations under slight tension while bowing and placing a lens haptic portion of said lens haptic under compression, said lens haptic configured to position and stabilize said lens optic within the anterior chamber of the eye; and
a connection portion located between said lens optic and said lens haptic, said lens optic and said lens haptic being configured to be separately inserted through the small incision in the eye, and then connected together by said connection portion to assemble the anterior chamber refractive correction lens device within the anterior chamber of the eye to then function in unison within the anterior chamber of the eye.

3. A lens according to claim 2, wherein said toric lens optic is defined by a toric lens surface located on one side of said lens optic.

4. A lens according to claim 2, wherein said multifocal lens optic is defined by a multifocal lens surface located on one side of said lens optic.

5. A lens according to claim 2, wherein said a toric lens surface is located on one side of said lens optic and a multi-focal lens surface is located on an opposite side of said lens optic.

6. A lens according to claim 2, wherein said multifocal lens optic is defined by a multifocal lens surface having at least two zones of different lens power.

7. A lens according to claim 6, wherein said multifocal lens surface includes a center circular surface portion and at least one concentric ring surface portion having different lens power.

8. A lens according to claim 1, wherein said lens optic is a wavefront lens optic.

9. A deformable anterior chamber refractive correction lens device, said device comprising:
a deformable anterior chamber lens optic configured to be inserted through a small incision in the eye into the anterior chamber of the eye and fit within the anterior chamber of the eye, said lens optic being a refractive, wavefront, and multifocal lens optic;
a deformable anterior chamber lens haptic configured to be inserted through a small incision in the eye into the anterior chamber of the eye and fit within the anterior chamber of the eye, said haptic portion configured for positioning said lens optic within the anterior chamber of an eye; and
a connection portion located between said lens optic and said lens haptic, said lens optic and said lens haptic being configured to be separately insert through a small incision in the eye and then connected together by said connection portion to assemble the functioning anterior chamber intraocular lens device.

10. A lens according to claim 9, wherein said wavefront lens optic is defined by a wavefront lens surface located on at least one side of said lens optic.

11. A lens according to claim 9, wherein said multifocal lens optic is defined by a multifocal lens surface provided on at least one side of said lens optic.

12. A lens according to claim 9, wherein said wavefront lens optic is defined by a wavefront lens surface located on one side of said lens optic and said multi-focal lens optic is defined by a multifocal lens surface located on an opposite side of said lens optic.

13. A lens according to claim 9, wherein said multifocal lens optic is defined by a multifocal lens surface having at least two zones of different lens power.

14. A lens according to claim 13, wherein said multifocal lens surface includes a center circular lens surface and at least one concentric ring lens surface having different lens power.

15. A lens according to claim 9, wherein said refractive correction lens includes a least one toric lens surface.

16. A deformable anterior chamber refractive correction lens device, said device comprising:
a deformable anterior chamber lens optic configured to be inserted through a small incision in the eye into the anterior chamber of the eye and fit within the anterior chamber of the eye, said lens optic being a refractive, multifocal, toric, and wavefront lens optic;
a deformable anterior chamber lens haptic configured to be inserted through a small incision in the eye into the anterior chamber of the eye and fit within the anterior chamber of the eye, said lens haptic configured for positioning said lens optic within the anterior chamber of an eye; and
a connection portion located between said lens optic and said lens haptic, said lens optic and said lens haptic being configured to be separately insert through a small incision in the eye and then connected together by said connection portion to assemble the functioning anterior chamber refractive correction lens device.

17. A lens according to claim 16, wherein said multifocal lens optic is defined by a multifocal lens surface provided on at least one side of said lens optic.

18. A lens according to claim 16, wherein said toric lens optic is defined by a toric lens surface provided on at least one side of said lens optic.

19. A lens according to claim 16, wherein said wavefron lens optic is defined by a wavefront lens surface provided on at least one side of said lens optic.

20. A lens according to claim 1, wherein said lens optic is also a toric lens optic.

21. A lens according to claim 1, wherein said lens optic is also a wavefront lens optic.

22. A lens according to claim 20, wherein said lens optic is also a wavefront lens optic.

23. A lens according to claim 1, wherein said lens optic is a bifocal lens optic and includes a center circular-shaped multifocal lens surface or zone having and add of +0.5 to +3.0 diopters (D) of approximately 3 millimeters (mm) and an outer concentric ring-shaped multifocal lens surface or zone for early to late presbyopes.

24. A method of making a refractive correction lens, comprising the steps of:
making a mold pin having a custom refractive, multifocal, toric and wavefront len optic surfaces thereon; and
molding the lens in a mold cavity fitted with said mold pin.
